(19) United States
(12) Patent Application Publication
(10) Pub. No.: US 2006/0008907 A1
(43) Pub. Date: Jan. 12, 2006

(54) Control of gene expression via light activated RNA interference

(75) Inventors: Simon H. Friedman, Kansas City, MO (US); Samit Shah, Kansas City, MO (US)

Correspondence Address: GREER, BURNS & CRAIN, 300 S WACKER DR, 25TH FLOOR, CHICAGO, IL, 60606, US

(73) Assignee: The Curators of the University of Missouri

(21) Appl. No.: 11/148,891

(22) Filed: Jun. 9, 2005

Related U.S. Application Data
(60) Provisional application No. 60/578,218, filed on Jun. 9, 2004.

(51) Int. Cl. A61K 48/00; C12N 15/85

(52) U.S. Cl. 435/455; 514/44

(57) ABSTRACT

A method for controlling the spacing, timing and degree of gene expression that includes selecting a target mRNA, obtaining siRNA corresponding to the target mRNA, modifying the siRNA with a photo-labile group to inhibit RNA interference, introducing the modified siRNA into a cell, and selectively irradiating the cell with light having a predetermined wavelength. A modified siRNA capable modulating gene expression that includes an siRNA that targets a predetermined complementary mRNA and at least one photo-labile group attached to said siRNA.

### BACKGROUND OF THE INVENTION

RNA interference (RNAi) is a recently described cellular phenomenon that has made a major impact in functional genomics, and is rapidly becoming an important method for analyzing gene functions in eukaryotes and holds promise for the development of therapeutic gene silencing. RNAi is a post-transcriptional process triggered by the introduction of double-stranded RNA (dsRNA), which leads to gene silencing in a sequence-specific manner. RNAi has been reported to naturally occur in organisms as diverse as nematodes, trypanosomes, plants and fungi. It most likely serves to protect organisms from viruses, modulate transposon activity and eliminate aberrant transcription products.

Short interfering RNA (siRNA) exist naturally in cells and degrade a target mRNA, thereby inhibiting gene expression of the gene corresponding the particular mRNA. More specifically, dsRNA is introduced into a cell, and an enzyme contained within the cell, DICER, degrades the dsRNA into small (21-23 nt) interfering RNA duplexes, which are the siRNA. An RNAi-induced-silencing-complex (RISC) within a cell incorporates siRNA, and the RISC/siRNA is then able to direct degradation of a target mRNA using a nuclease activity associated with the RISC/siRNA.

Separately, photo-caging has been used as a method to control gene expression in applications involving steroid hormones, plasmid DNA, mRNA and has also been used to block the 2′OH of a ribozyme to permit light control of the ribozyme targeted RNA degradation.

### SUMMARY OF THE INVENTION

The invention provides for a modified siRNA and a method of controlling the spacing, timing and degree of gene expression that includes the modified siRNA.

Generally, the siRNA is modified with a photo-labile group that may be selectively cleaved to modulate expression of a target mRNA. The method includes selecting a target mRNA, obtaining or creating siRNA corresponding to the target mRNA, modifying the siRNA with a photo-labile group, such as DMNPE, transfecting the modified siRNA into a cell, and irradiating the cell with ultraviolet light, preferably wherein the ultraviolet light has a wavelength greater than 320 nm. Various embodiments of the invention include modifying the siRNA in one of a plurality of different manners. For example, in one embodiment, a backbone phosphate of the siRNA is modified with the photo-labile group. In other embodiments, the siRNA is modified at the 3′ and or 5′ hydroxyl group of the siRNA with the photo-labile group, modified at the 3′ and/or 5′ phosphate group of the siRNA with the photo-labile group, or a photo-labile group is provided to form a cleavable linker between two nucleotides in the siRNA chain. Embodiments of the invention also include conjugation of the photo-labile group to other molecules including peptides or proteins that confer other properties, such as steric bulk or improved membrane transport ability, to the siRNA.

### BRIEF DESCRIPTION OF THE DRAWINGS

FIG. 1 is a schematic diagram illustrating RNA interference by dsRNA and small interfering RNA (siRNA);

FIG. 2 is a schematic diagram illustrating a hormone caging approach for light control of gene expression;

FIG. 3 are sequences of a target (GFP) and control sequences of siRNA for a test system;

FIG. 4 illustrates modification of siRNA duplex with DMNPE groups;

FIG. 5 is a chart illustrating the influence of siRNA on GFP expression in HeLA cells in the presence and absence of light exposure;

FIG. 6 is a graph illustrating decreasing GFP signal with increasing light exposure in cells treated with caged target siRNA;

FIG. 7 is a chart illustrating that decreasing amounts of DMNPE in caging reaction with siRNA leads to decreasing caging and increasing ease of release of active siRNA;

FIG. 8 is a schematic diagram illustrating some of the iterations for photo-labile group attachment;

FIG. 9 illustrates commercially available precursors to make a series of photo-labile protecting groups of varying size;

FIG. 10 illustrates a commercially available precursor to allow fine-tuning of bulk through amine additions;

FIG. 11 illustrates a 5′ anti-sense phosphate addition shown to limit RNA interference (top) and photo-labile version (bottom);

FIG. 12 illustrates acylation of photo-labile amine linker to introduce increased steric bulk;

FIG. 13 is a schematic diagram illustrating a caging strategy targeting of the 5′ phosphate on the anti-sense strand of siRNA;

FIG. 14 is a graph illustrating fluorescent signals of the caging strategy illustrated in FIG. 13 and that of a GFP plasmid;

FIG. 15 illustrates increasing bulk on 5′ phosphate photo-labile linker by conjugation using a hydralink system;

FIG. 16 is a schematic diagram illustrating blocking 5′ phosphate with proteins that can both block binding to the RISC and enhance uptake of siRNA;

FIG. 17 is a schematic diagram illustrating a caging strategy targeting the 5′ phosphate on the anti-sense strand of siRNA with different modifications and increasing bulk;

FIG. 18 is a graph illustrating the effect of 5′ p c biotin modified siRNA on GFP expression;

FIG. 19 is a graph illustrating the effect of 5′ p c biotin-avidin modified siRNA on GFP expression;

FIG. 20 is a graph illustrating the effect of 5′ pc biotin modified siRNA on GFP expression normalized to RFP;

FIG. 21 is a graph illustrating the effect of 5′ pc biotin modified siRNA on GFP expression normalized to RF″P in the presence of avidin;

FIG. 22 illustrates that addition of phosphate to 5′ OH of anti-sense strand of siRNA activates it for RNA interference;

FIG. 23 illustrates photoprotection of 5′ OH to prevent activation of siRNA via phosphorylation by endogenous kinase;

FIG. 24 illustrates pseudo-orthogonal photo-protecting groups;

FIG. 25 is a schematic diagram illustrating differential caging, leading to differential control of two genes;

FIG. 26 illustrates 2′OH photo-protected phophoramidite synthesis;

FIG. 27 illustrates increasing bulk of 2′OH photo-labile group for incorporation into phophoramidite synthesis;

FIG. 28 illustrates addition of amine handle to 2′OH photo-labile group to allow further modification post synthesis;

FIG. 29 is a schematic diagram illustrating sense strand photo-cleavable linker approach;

FIG. 30 illustrates a modeled photo-cleavable spacer in duplex;

FIG. 31 is a schematic diagram illustrating patterning setup;

FIG. 32 is a schematic diagram illustrating a hairpin linking the sense and anti-sense strands with a photocleavable linker;

FIG. 33 is a graph illustrating the fluorescent signals of the caging strategy illustrated in FIG. 32 and that of a GFP plasmid;

FIG. 34 is a graph illustrating fluorescent signals following delivery of a large concentration of the highly caged siRNA;

FIG. 35 is a schematic diagram illustrating a caging strategy targeting key positions on the siRNA using phosphorothioate chemistry;

FIG. 36 is a graph illustrating the fluorescent signals of the strategy illustrated in FIG. 35 and that of a GFP plasmid;

FIG. 37 is a schematic diagram illustrating a caging strategy wherein siRNA is modified with the [7-(diethylamino)coumarin-4yl]methyl caging group;

FIG. 38 is a graph illustrating the fluorescent signals of the caging strategy illustrated in FIG. 37 and that of a GFP plasmid;

FIG. 39 is a graph illustrating the fluorescent signals of the caging strategy illustrated in FIG. 37 and that of a GFP plasmid;

FIG. 40 is a graph illustrating the fluorescent signals of a caging strategy involving caging dsRNA that needs to be processed by both Dicer and RISC before exhibiting RNAi effect;

FIG. 41 is a schematic diagram illustrating caging of either the sense or anti-sense strand alone; and

FIG. 42 is a graph illustrating the fluorescent signal of the caging strategy illustrated in FIG. 41 an that of a GFP plasmid;

### DETAILED DESCRIPTION OF THE INVENTION

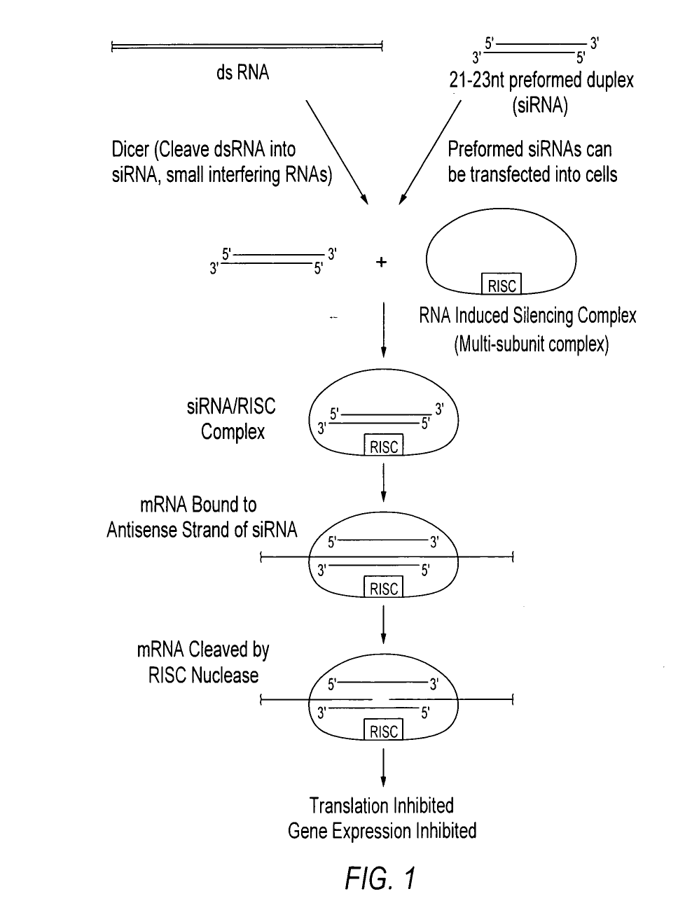

RNA interference is a recently described cellular phenomenon whereby double-stranded RNA specifically initiates the destruction of a target mRNA, and thereby affects the expression of the protein for which the mRNA codes. As illustrated in FIG. 1, double stranded RNA is processed in the cell by the ribonuclease, Dicer, to make small duplex RNA pieces of 21-23 nucleotides, containing a 2 base 3′OH overhang. These duplexes are then recognized and bound by a protein complex named the RNA induced silencing complex (RISC). Upon forming this assembly, the RNA/RISC can then bind to a single stranded mRNA that is complementary in sequence to the anti-sense strand of the duplex RNA. After binding, the mRNA is degraded by a nuclease activity associated with the RISC, leading to reduced gene expression.

Although large double stranded RNAs can be used to initiate this process, small preformed 21-23 base RNA duplexes introduced into cells also accomplish this effect. These are named small interfering RNAs, or siRNA. siRNAs have proven to be a very useful tool to examine the effect of gene expression, as they are able to “knock-down” expression of genes in a very specific fashion. In addition, they generally show greater efficiency, and produce effects that are longer lived than those found with the anti-sense approach.

Photo-caging has been used as a method to control gene expression in applications involving steroid hormones, plasmid DNA, mRNA and has also been used to block the 2′OH of a ribozyme to permit light control of the ribozyme targeted RNA degradation.

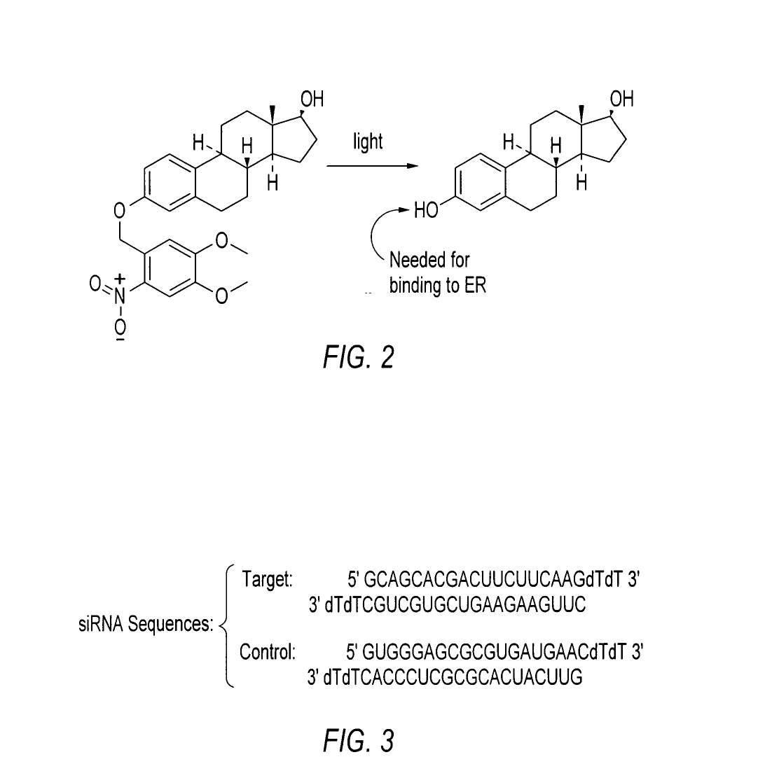

For example, as illustrated in FIG. 2, in steroid hormone applications, caging with photo-labile groups (PLG) has been used as a method for temporarily blocking intermolecular interactions. Specifically, a phenolic hydroxyl group of estradiol was caged using 2-nitroveratryl-bromide. This additional group putatively blocks an interaction between estradiol and estradiol receptor (ER), preventing the ER from binding to its target DNA sequence. Upon irradiation, the protecting group is cleaved, exposing the phenolic OH, which then allows for binding to the ER, concomitant binding of the now-active ER to its target DNA sequence, and the subsequent activation of transcription of the gene that follows the DNA binding site. Using luciferase as the target gene, this allowed for convenient assessment of transcription activation. However, by their nature, steroid hormones are only capable of targeting a limited number of receptors. Moreover, the receptors targeted by steroid hormones target a binding site on DNA that is involved in the activation of many genes, and as such, these applications do not allow for a high degree of specificity. Thus, applications involving steroid hormone caging are potentially limited from general analysis of the consequence of gene expression in “normal” cells and organisms.

Another approach that has been used is that of photo-caging whole plasmids, wherein a whole plasmid containing the gene for green fluorescent protein (GFP) was reacted with 1-(4,5-dimethoxy-2-nitrophenyl) diazoethane (DMNPE), which caged a small percentage of the phosphate diester linkages of the plasmid. The apparent aim of this approach was to block transcription of the plasmid after introduction into a cell through interference of the caging groups with the transcription machinery. Caged plasmids were transfected into HeLa cells, and it was demonstrated that exposure to light resulted in an increase in GFP signal relative to unexposed cells. However, these applications also displayed significant signs of photo-toxicity.

A related approach is the use of caging to block the phosphodiester linkages of mRNA. The aim of this approach is to block interaction of the nucleic acid with its target enzymes, in this case the translation machinery. A 6-bromo-7-hydroxycoumarin-4-methyl group is used to block a small proportion of the phosophodiester linkages of a 1 kb mRNA coding for the GFP protein. This was sufficient to provide a measure of photo-control to the expression of GFP, i.e. light exposure resulted in an increase in the amount of GFP expressed in a cell-free translation system. This approach also showed effectiveness in whole organisms (zebrafish), although it required direct injection into the cells of the organism.

Again, while this approach demonstrated the utility of blocking of phosphodiester linkages by photo-labile protecting groups, and showed how this could effectively prevent interaction with the target translation machinery, the limitation of this approach is that it can only be used with exogenously delivered genes, which is a severe limitation when studying the effect of endogenously expressed genes.

As an alternative to these approaches for photo-controlled gene expression, the instant invention includes “light activated RNA interference” or “LARI,” which uses siRNAs that incorporate appropriate photo-labile groups to modulate the RNA interference effect until light exposure. siRNA is an especially advantageous candidate for use as the basis for controlling gene expression with light for several reasons. First, research is demonstrating that siRNA is a very general, very robust method for the control of gene expression, more so than anti-sense or ribozyme methods. It has been suggested that for every gene there is an effective siRNA, which means that essentially any gene can be targeted efficiently via siRNA. As such, it forms an ideal foundation on which to introduce light control. In addition, siRNAs control the expression of endogenous genes, something that the plasmid and mRNA approaches described above cannot do.

As discussed, short interfering RNA (siRNA) exist naturally in cells and are short 21-23 base RNA duplexes with 2 base 3′ overhangs that target specific sequences of mRNA for degradation, thereby inhibiting gene expression of the gene corresponding the particular mRNA. The siRNA are derived when double stranded RNA (dsRNA) is introduced into a cell, and an enzyme contained within the cell, DICER, degrades the dsRNA into small (21-23 nt) interfering RNA duplexes, which are the siRNA. The anti-sense strand of the siRNA/RISC complex recognizes the complementary mRNA, which is then degraded by a nuclease activity associated with the RISC.

The instant invention includes an siRNA that has been modified by a photo-labile group in a predetermined manner, and also provides method of controlling the spatial and temporal expression patterns of genes by modulating the activity of the siRNA. This is done by first selecting a target mRNA and either obtaining or creating a corresponding siRNA. The siRNA is then modified with a photo-labile group in a predetermined manner, such as by “caging” the siRNA with a photo-labile group, such as 4,5 dimethoxy-2-nitrophenyl ethyl (DMNPE). The photo-labile group modified siRNA, or “caged” siRNA, e.g., siRNA/DMNPE, is then transfected into a cell. Modifying the siRNA, such as by caging, diminishes the ability of the siRNA to degrade the target mRNA, and thus the mRNA is available to participate in translation (and eventually expression) of the encoded gene. It is believed that “caging” may potentially eliminate entirely the ability of the siRNA to degrade target mRNA.

When the cell is subsequently irradiated with light, preferably longer wavelength UV light, such as between 300 nm and 400 nm, DMNPE is cleaved, leaving the native, natural siRNA free to associate with RISC and subsequently degrade the target mRNA. Thus, by selectively irradiating the cells containing the siRNA/DMNPE, expression of the gene encoded by the target mRNA may be selectively modulated.

Potential applications are widespread and voluminous, including uses for developmental biologists to control location, spacing, timing and the amount of gene expression. Other uses include gene therapy, e.g., down-regulating gene expression in a specific area or zone of the body. Another application is tissue engineering, by patterning expression in cells, allowing cell level control of different genes, with the potential for engineering their interactions. Still another application is nanotechnology, such as information storage and networking at a cellular level.

More specifically, the instant invention demonstrates a method wherein gene expression is modulated in a light-controllable fashion using photo-caged siRNA. Embodiments of the invention provide a chemical approach that promotes the generic spatio-temporal control of gene expression. The general applicability of RNA interference has shown to be valuable for controlling gene expression. In addition, control of gene expression via light activated RNA interference allows the modulation of endogenous genes, whereas mRNA and plasmid approaches do not.

Embodiments of the invention include modulating gene expression using siRNA that has been modified, or “caged,” with a photo-labile group. It is contemplated that caging of the siRNA may proceed using any strategy that results in a functional siRNA following cleavage of the photo-labile group, and it is additionally contemplated that a virtually limitless number of caging moieties may be used, providing the caging moieties sufficiently inhibit siRNA activity. While the invention should not be construed as being limited to the exemplary caging methods and caging moieties discussed herein, several examples will be provided to illustrate the invention and various embodiments thereof.

More specifically, caging of siRNA may proceed via a plurality of caging techniques, which include but are not limited to 1) modification of the phosphate backbone of siRNA with a photo-labile group; 2) modification of the 3′ and/or 5′ hydroxyl groups of siRNAs with PLGs; 3) modification of the 3′ and/or 5′ phosphate groups of siRNAs with PLGs; 4) modification of the 2′ OH groups of siRNA with PLGs; 5) replacing nucleotides in one or both of the strands of siRNA with a PLG; 6) the modifications described in (1)-(4) above, wherein the PLGs are further conjugated to other molecules including peptides or proteins which confer other properties such as steric bulk (to further block RNA interference) or membrane transport ability; 7) designing a hairpin by linking the sense and antisense strands with a PLG; 8) delivering a large concentration of the highly caged siRNA to the cell(s); 9) targeting key positions on the siRNA using phosphorothioate chemistry; 10) targeting 5′ OH on the antisense strand using 4,5 dimethoxy-2-nitrobenzyl chloroformate; 11) modifying siRNA with the [7=(diethylamino)coumarin-4yl]methyl caging group; and 12) modifying siRNA with 6-bromo-4-diazomethyl-7-hydroxycoumarin (Bhc-diazo). Ortho-nitrobenzylic based caging groups were used for strategies 1 through 9, while alternative caging groups, namely [7-(diethylamino)coumarin-4yl]methyl caging group and 6-bromo-4-diazomethyl-7-hydroxycoumarin (Bhc-diazo), were used for strategies 10 and 11, respectively.

Experiments and Results

To illustrate the invention, a system using 96 well plates was established for the analysis of RNA interference. A green fluorescent protein (GFP)/HeLa system permits the use of a scanning micro-plate fluorescence reader to assess the inhibition of expression of GFP by siRNAs. Briefly, plasmids encoding the gene for GFP are transfected into HeLa cells, cultured onto 96 well black-walled culture plates. Included in this transfection are siRNAs if required by the experiment. After six hours of transfection, the plates are either light exposed or masked. After 42 hours, culture media is removed and replaced with buffer, and the plate scanned in the fluorescence reader using an excitation filter of 485 nm and an emission filter of 535 nm. Cells transfected with GFP plasmids show significantly higher fluorescent signals in comparison with mock-transfected cells (typically ˜40,000 counts versus ˜1800). This then is the signal that is monitored for changes due to RNA interference and/or light exposure.

Data is expressed as actual fluorescence signals, not signals that have been normalized to the expression of another gene. Using 96-well plates permits averaging of multiple wells for each experimental condition (5 to 8 points), allowing use of the absolute signal with low standard errors. This, in turn, provides control for the effect of potential photo-toxicity more easily.

The gene encoding for GFP has been successfully targeted using siRNA. The GFP target sequence is illustrated in FIG. 3, as well as a control siRNA sequence. The target siRNA significantly reduces expression of GFP via RNA interference, whereas the control sequence does not. In addition, this is not due to an anti-sense effect. Because this system is well characterized, it makes an ideal test system for investigating the modulation of RNA interference. The adaptation for use with a 96-well plate reader allows for rapid analysis of numerous experimental iterations of the invention. While the instant invention contemplates modifying the siRNA in any manner that achieves the desired level of gene expression, several exemplary methods of modification are provided. In addition, while a plurality of caging moieties are contemplated for use with the invention, for purposes of illustration, a few exemplary caging groups will be discussed herein, namely an ortho-nitrobenzylic based caging group (1-(4,5-dimethoxy-2-nitrophenyl) diazoethane (DMNPE)), [7-(diethylamino)coumarin-4yl]methyl caging group (DEACM), 6-bromo-4-diazomethyl-7-hydroxycoumarin (Bhc-diazo), 2-nitrobenzyl, 4,5 dimethoxy 2-nitrobenzyl, Alpha-carboxy-2-nitrobenzyl, 1-(2-nitrophenyl)ethyl nitroindoline, 4-methoxy 7-nitroindoline, 1-acyl 7-nitroindolines, 1-(2-nitrophenyl)ethyl ethers of 7-hydroxycoumarins, 7-(alkoxy coumarin-4yl)methyl esters, 6,7-(dialkoxy coumarin-4yl)methyl esters, 6-bromo-7-(alkoxy coumarin-4yl)methyl esters, 7-dialkylamino (coumarin-4yl)methyl esters, p-hydroxyphenacyl, and 6-bromo-7-hydroxycoumarin-4-ylmethyl. However, the invention is not intended to be limited to these few caging groups, but is contemplated to encompass any caging group that would achieve favorable caging of a target siRNA.

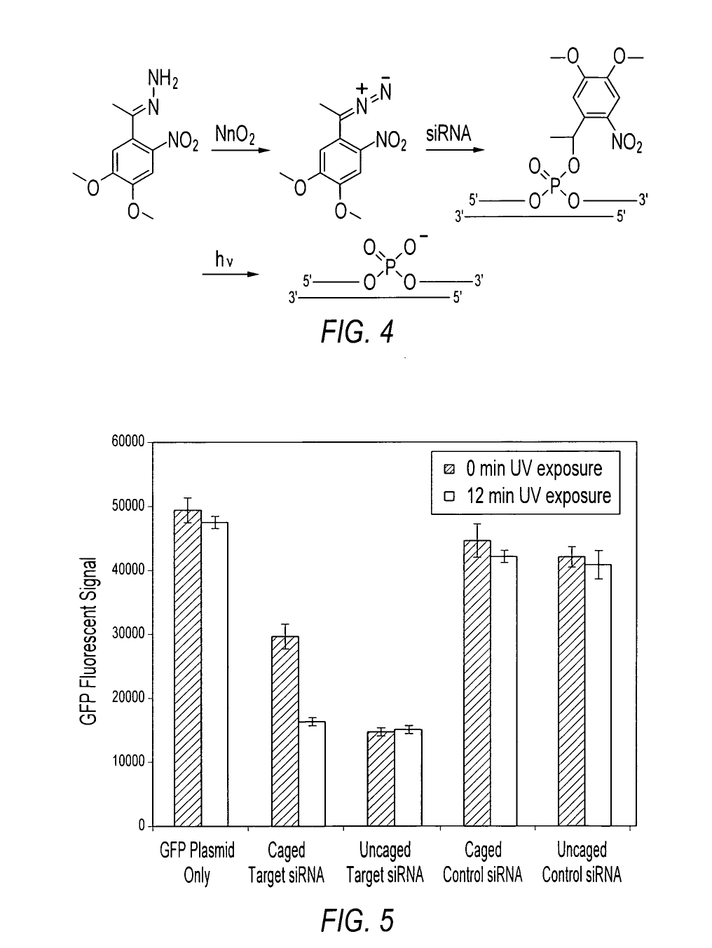

Caging is effected by reaction of a precursor hydrazone to create a corresponding diazo compound, which can be reacted with phosphate groups on nucleic acids (FIG. 4). A GFP targeting siRNA was used, allowing for the quantification of gene expression via the fluorescent signal generated by GFP. GFP targeting siRNA has also been well characterized, eliminating issues such as an anti-sense effect. To demonstrate the efficacy of the invention, two siRNAs are used experimentally: the target siRNA that directs the degradation of GFP mRNA, and a control siRNA that has been shown to have no effect on GFP expression.

RNA oligonucleotides may be obtained commercially, and subsequently deprotected and annealed using standard protocols. One nmole of both target and control duplexes were photo-protected using a ˜100 fold excess of diazo reagent. Subdued lighting was used for all manipulations. After protection, siRNAs were precipitated and extracted twice with chloroform. The extent of photo-caging was determined using the absorbance of the sample at 355 nm and the extinction coefficient for the caged DMNPE group. Under the conditions described, 1.4 photo-caging groups per duplex were obtained. This 3% caging efficiency is similar to the results found in two other systems. The target caged and uncaged duplexes were further characterized by determining their melting temperatures (Tm). Both uncaged and caged siRNAs showed the expected broad transitions with Tm values of 54.3° C. and 65.1° C. respectively. It is possible that the quenching of phosphate charge via the DMNPE group is the source of the relative stabilization of the caged siRNA.

Determination of an appropriate filter is an important consideration owing to the detrimental effects of phototoxicity, as there exists a potential for a toxic effect of the UV radiation required to deprotect the DMNPE groups. While it is contemplated that embodiments of the invention may be used in conjunction with a variety of appropriate filters, one exemplary filter that permits the transmission of the frequencies needed for deprotection (>320 nm) but blocks the shorter and more toxic wavelengths is the WG-320 320 nm longpass filter. The samples were exposed for 12 minutes using a Blak-Ray fluorescent UV lamp (XX-15L 15w) at a distance of 10 cm. After light exposure, the culture media was again changed and the cells were allowed to culture for an additional 42 hours. At that time GFP signal was quantified in vivo using microplate fluorescence. The fluorescent signals from five wells were averaged for each experimental condition. Normalizing for transfection efficiency by using an internal standard was not necessary. To accommodate the normal variation that results from variations in transfection efficiency, each experimental point is an average of five wells. In addition, all the points represented in a figure come from a single 96 well plate, another factor that adds to more consistent transfection efficiencies. This consistency is evidenced by the relatively low standard errors observed.

Using the above described materials and methods, it is possible to demonstrate that only target siRNA protected with photo-labile groups show a light-dependent change in GFP expression. For example, a comparison of six sets of experimental conditions may be analyzed on a 96 well plate: 1) transfection with GFP plasmid only, to determine the maximum expected fluorescent signal and to determine if light exposure affected it. This is a probe of photo-toxicity; 2) transfection with GFP plasmid and un-caged target siRNA, to determine the maximum expected RNA interference effect; 3) transfection with GFP plasmid and caged target siRNA, to determine the variation of the RNA interference effect with light exposure; 4) transfection with GFP plasmid and un-caged control siRNA, to insure that any reduction in GFP signal by the target siRNA is due to its specific sequence; 5) transfection with GFP plasmid and caged control siRNA, to insure that any reduction in GFP signal by the target siRNA is not due to toxicity of the photo-released DMNPE groups; and 6) mock transfection using transfection agent but no plasmid or siRNA, to insure that auto-fluorescence of the cells (and changes in this signal upon light exposure) does not significantly alter results.

Ten wells were used for each condition, five with light exposure, five without. Cultured cells were transfected with the requisite GFP and/or siRNA using lipofectamine. After six hours of transfection, the transfection mixtures were removed, fresh media was added, and the cells either exposed to light for twelve minutes or masked. After exposure, the media was again changed and the cells returned to the incubator for a further 42 hours of culturing. This amount of time results in a maximum RNA interference effect. The media was then replaced with buffer and read the GFP signals in the micro-plate fluorescence reader.

Results are summarized in FIG. 5. The only cells that had a significant difference in GFP expression upon exposure to light were the cells that had been treated with the caged target siRNA (p<0.005). Selective phototoxicity can be eliminated as a mechanism of action because light had no significant effect on GFP signal in any of the other samples. Light induced damage of the RISC can be eliminated because the non-photoprotected target siRNA gave equivalent suppression of GFP signal with and without light. Finally, selective toxicity of the released DMNPE group can be eliminated as a mechanism of action because the caged control siRNA gave equivalent GFP signal with and without light. The most reasonable interpretation of the results is that the DMNPE group blocked RNA interference and that light exposure released active siRNA.

The results are consistent with the invention, in that modification of siRNA with photo-labile groups allows the modulation of RNA interference by exposure to light. The deprotected siRNA is fully active as compared with the unprotected siRNA. However, the caging of the protected siRNA is not complete, i.e. there is still some residual RNA interference even in the caged target siRNA. This is reasonable given the caging amount of 1.4 groups per duplex. It is reasonable to assume that modification of some positions on the duplex will be more effective than others at blocking RNA interference.

Embodiments of the invention are not limited to gene expression or lack thereof, but also contemplate modulating the degree of RNA interference and resulting expression. The principle may be illustrated using the same system as above. This is an additional potential benefit of caging, as it will allow the variation of the amount of expression of a given gene in target tissue.

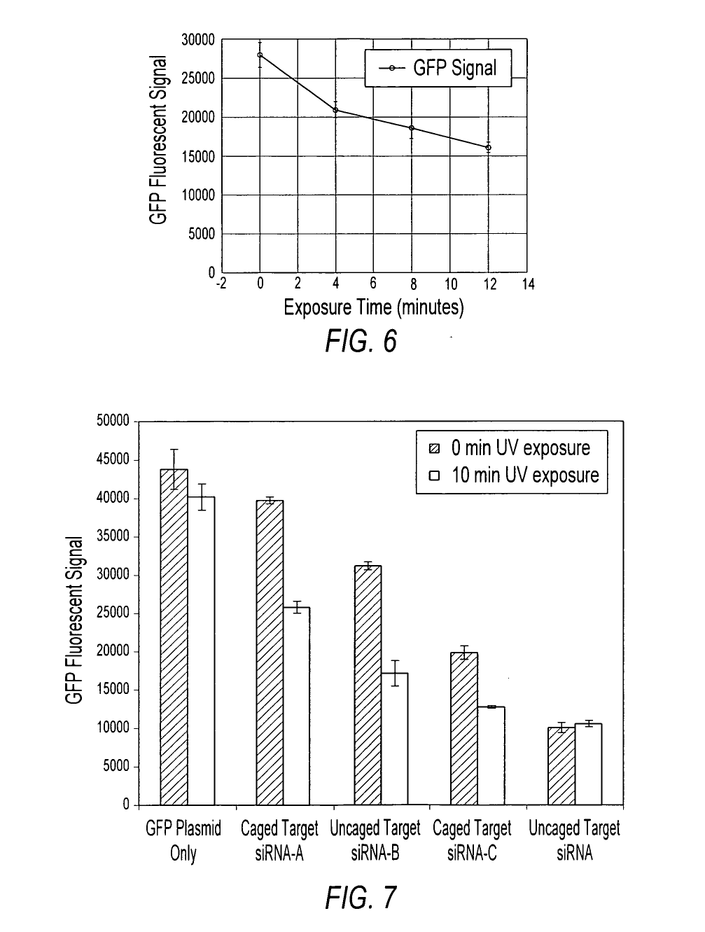

To do this, expression of GFP in cells that had been treated as before with caged target siRNA were analyzed. Light exposure during DMNPE deprotection however was varied from 0 to 12 minutes. FIG. 6 summarizes the results of this experiment. Increasing exposure of light gave a gradual increase in the RNA interference effect between the two limits established previously.

These results indicate that RNA interference can be brought under the control of light through the use of photo-labile groups, i.e., that light control of RNA interference is possible. The modification of siRNA with a photo-labile group results in a species that is blocked from full RNA interference until irradiated, whereupon it is as active as unprotected siRNA. In addition, this effect can be modulated by varying the amount of time that photo-deprotection takes place. The positional and steric factors that may allow for a complete caging of the siRNA are also an area of interest within the context of the invention, utilizing the growing understanding of the structural features required for effective RNA interference. This approach may be useful for a range of biological studies, in particular studies of development, where the spacing, timing and amount of gene expression are key factors in determining developmental outcome.

The caging of the siRNA does not completely abrogate RNA interference, likely owing to the 3% caging efficiency corresponding to 1.4 phosphate groups modified on average per duplex. It is probable that some duplexes are completely unmodified, while others are modified in positions that are not able to block the siRNA/RISC interaction. The inventors believe that a potential solution to this problem is to increase the substitution level, resulting in a larger proportion of the siRNA duplexes that have blocked positions.

Thus, systematically increasing the number of equivalents of the DMNPE reagent used during caging has addressed the problem. Specifically, in one experiment, 175, 875 and 1750 mole equivalents were used to modify three different samples. Actual concentration of oligonucleotides used were measured by UV absorbance after modification and precipitation. In general the actual concentration is somewhat lower than the nominal, possibly due to precipitation efficiencies. Monitoring actual oligonucleotides concentrations allows for a more precise determination of molar ratios of reactants.

As illustrated in FIG. 7, the method of analysis of the siRNAs caged with increasing amounts of DMNPE groups was identical to the previously described analysis of modified siRNA. Again, the only samples that showed a statistically significant difference in GFP signal upon irradiation were those treated with modified siRNAs (p<0.05). With increasing amounts of caging reagent there is a greater blocking of RNA interference before irradiation. This is paralleled by a greater resistance to complete release of fully active siRNA with irradiation. In theory, increasing the time of irradiation could lead to a complete release of active siRNA. However, expanding irradiation beyond 10-12 minutes leads to a reduction in control GFP expression that is significant in comparison with cells that are not irradiated.

It therefore appears that simply increasing the amount of caging may be insufficient to create a system that is switchable from no RNA interference to full RNA interference because it leads to a substitution level that requires toxic levels of light to remove. Specific steric and positional effects of modification should prove helpful, as the inventors believe that the siRNA/RISC complex is like any other ligand/protein complex in that it is driven by specific interactions of the ligand with specific functionalities on the protein. Modification of those crucial contact points by photo-labile groups should allow the complete blocking of RNA interference until irradiation releases the fully active species.

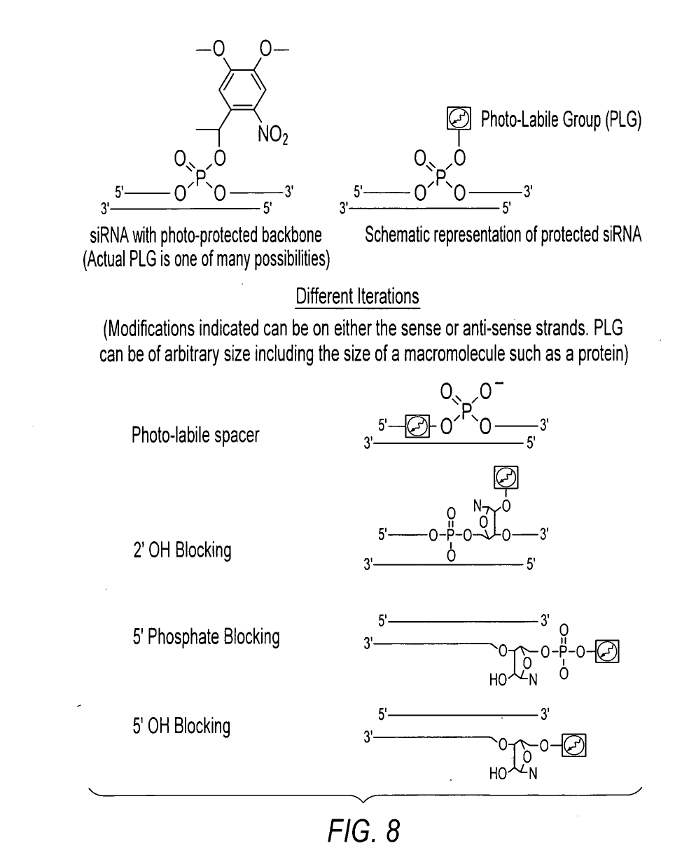

The invention encompasses modulation of RNA interference based on understanding the structural features that permit siRNA to bind to the RISC and then making RNA interference a light activated phenomenon. As demonstrated above, blocking the interactions of the siRNA backbone in a random fashion with a DMNPE group is effective at temporarily blocking the RNA interference effect. Additionally, the invention includes determining the most effective positions for modification that will then allow RNA interference to be switched from completely off to completely on upon light exposure. Some of these variations are illustrated in FIG. 8.

Generally, reaction of an siRNA duplex with a caging compound, such as DMNPE, creates a species that is as active as unmodified siRNA after light exposure. However, the “caging” of the siRNA may be incomplete. In other words, the caged siRNA still shows some RNA interference effect, indicating that the modifications do not completely block interaction with the RISC, or other key macromolecules in the RNA interference pathway. Simply adding more caging groups does not appear to solve the problem: While the RNA interference effect can be completely blocked using more groups, it results in a species that requires toxic levels of light exposure to release fully active siRNA.

Accordingly, the invention contemplates an alteration of the already demonstrated approach, which is to add steric bulk to the random caging group. If these random backbone modifications act by blocking interactions between the siRNA and the RISC, then it follows that increasing the size of the groups will increase the level of the steric clash between the siRNA and RISC. Sites that were previously modified in a random fashion that had no clash will now potentially be too bulky to allow effective formation of the complex. Thus, one embodiment of the invention contemplates variation of the DMNPE group to introduce this steric bulk.

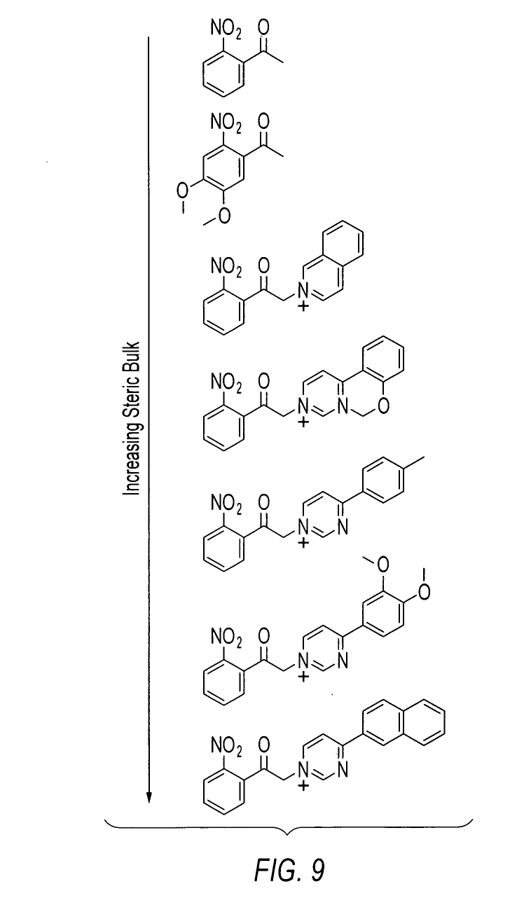

While the invention contemplates numerous caging moieties, DMNPE is especially advantageous. The DMNPE group contains the key features needed for photo-lability: a benzylic oxygen that is ortho to a nitro group. This arrangement allows for photo activation of the group and cleavage of the oxygen-benzylic carbon bond. As previously described, the starting material for the caging reaction is the ketone, which is converted to the hydrazone. This then is converted to the diazo form using MnO₂. This reactive species can then react with an oxygen nucleophile from a range of sources: an alcohol, a carboxylic acid, or in the case of caged siRNA, a phosphate group. The presence of the di-methoxy functionalities increases the wavelengths that the group can be deprotected with (as high as 340 nm), but the non-methoxylated version can still be deprotected with long UV (i.e. 320 nm). There are numerous commercially available compounds that contain this arrangement of functionalities but have greater steric bulk. FIG. 9 illustrates a sampling of these, with increasing steric bulk. Like the ketone precursor of DMNPE, these will be converted to the hydrazone, then to the diazo form, and ultimately reacted with the siRNA to form the caged form. As before, the inventors believe that the duplex remains formed by determining melting transition temperatures.

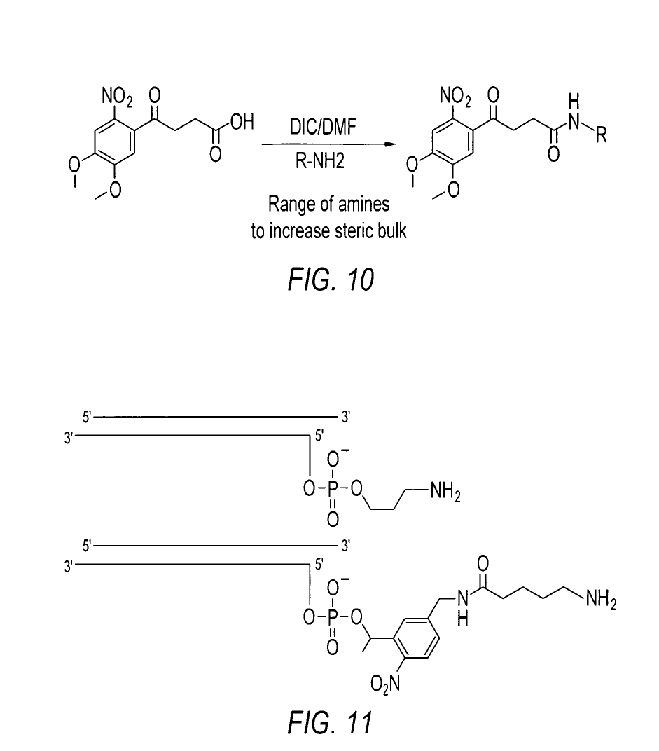

The invention anticipates that in the limit, there will be modifications that will be too bulky to even allow duplex formation. This process then will be one of a steric titration: adding sufficient bulk to more effectively block siRNA/RISC interaction while not so destabilizing the duplex to prevent duplex formation. An additional commercially available precursor that will be effective in this process is shown in FIG. 10. It too contains the nitro ortho to a ketone needed for the creation of an effective photo-labile group. In addition, it contains a synthetic handle, the carboxylic acid, that will allow straightforward elaboration to increasingly bulky groups, through acylation with a highly diverse set of commercially available amines. This compound will form the basis for a large library of caging compounds, with increasing steric bulk. This will allow the steric optimization to be performed in small increments, thereby increasing the likelihood of success.

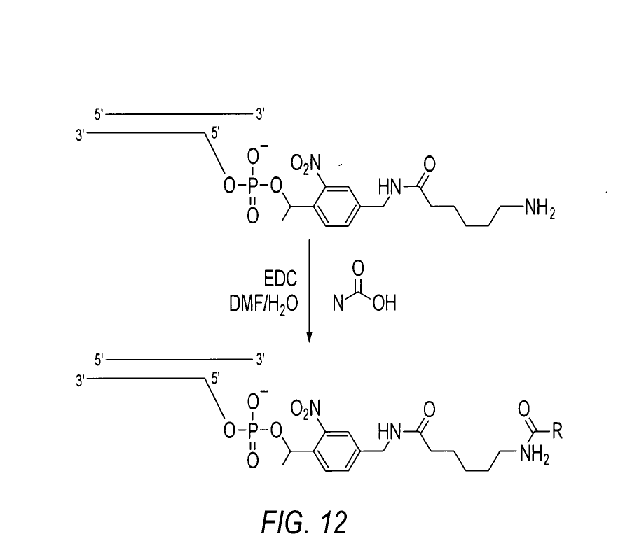

Another embodiment of the invention includes specific blocking of a 5′ phosphate. There may simply be limits to the effectiveness of random blocking of backbone phosphate groups in siRNA, even with the increase in steric bulk described above. Presumably, there are specific sites on the siRNA that are more important for making contact with the RISC, and therefore specifically blocking them should allow a much more effective and reliable caging of the siRNA (i.e. 100% blocking of RNA interference effect until light exposure. Modifying the 5′ phosphate on the anti-sense strand of siRNA with an amino containing linker results in a species that has limited ability to cause RNA interference. A simple extension of this is to place a photo-labile linker between the 5′ phosphate and this identical linker (FIG. 12). Fortunately, this photo-labile linker is commercially available and can be incorporated into RNA synthesized on the solid phase.

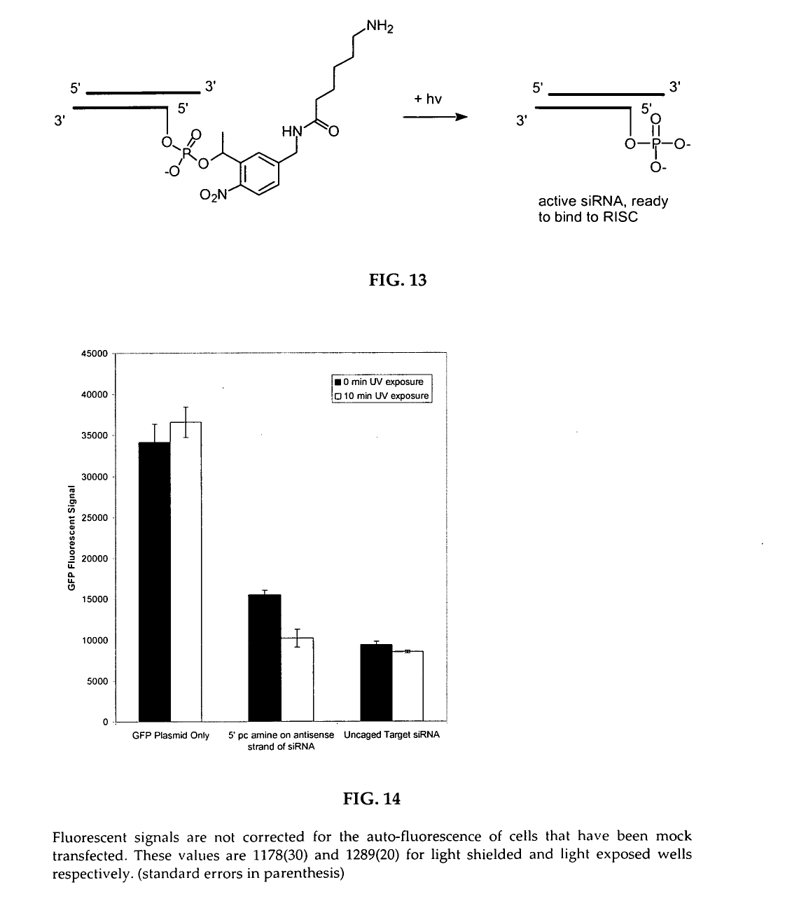

As illustrated in FIGS. 13 and 14, a 5′ phosphate on the antisense strand of the siRNA duplex is necessary for effective siRNA function, suggesting that cells confirm the authenticity of siRNAs and allow only bona fide siRNAs to silence the target gene. This was further corroborated by demonstrations that modifications on the 5′ phosphate of the antisense strand of siRNA abolished its ability to cause RNA interference. Since two photolabile groups could be removed with levels of light that were not toxic, it was reasoned that one could place a photolabile group on the 5′ end of the antisense strand of siRNA, rendering the siRNA inactive in the absence of light. Irradiation with light should result in an siRNA with a free 5′ phosphate on the antisense strand resulting in destruction of the target mRNA. A photocleavable amine modification on the 5′ end of the antisense strand was used for this study.

There is still some ambiguity concerning the absolute requirement of a free 5′ phosphate on the antisense strand for effective gene silencing. Some more recent studies describe sequences that exhibit effective gene silencing even with modifications on the 5′ antisense phosphate. It is believed that the nature of modification and incorporation of the correct strand into RISC may be critical in determining the absolute requirement of a free 5′ phosphate on the antisense strand of siRNA. Besides, assuming that there exists a binding equilibrium between RISC and siRNA, it is possible that siRNAs with 5′ modifications might be processed by RISC, although not as effectively as siRNAs having a free 5′ phosphate.

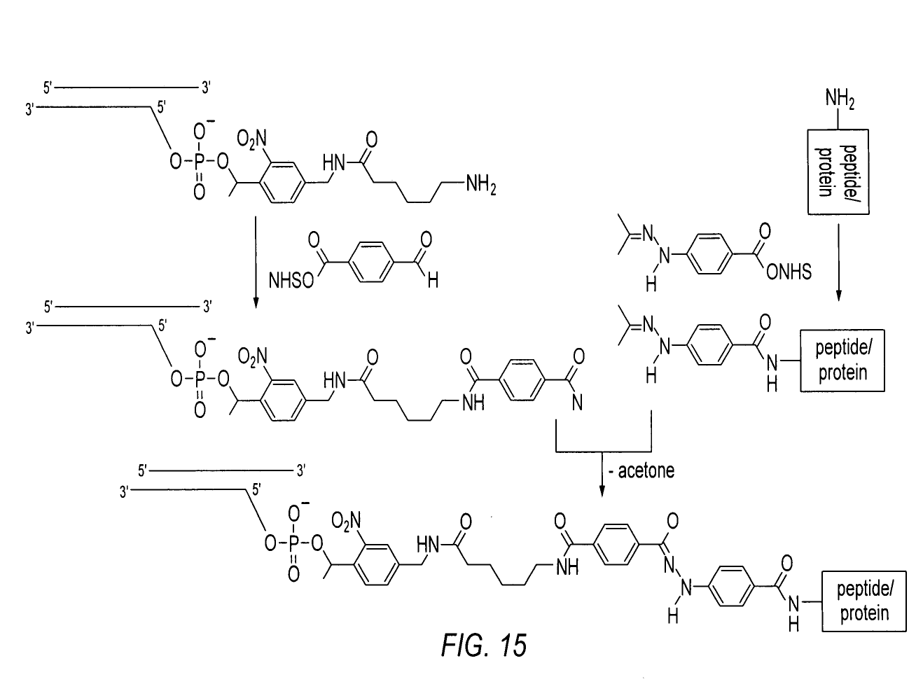

If this simple modification is not effective at completely blocking RNA interference, then increasing the caging of the siRNA by increasing the steric bulk of substituents added to the amino group via acylation may address the problem. Simple acids can be activated with the water soluble carbodiimide EDC, and conjugated directly to the amino terminus (FIG. 15). Of course the greatest steric bulk will be that introduced by a macromolecule, such as a protein or peptide. These can be incorporated onto the amino 5′ phosphate linker with a range of conjugation methods.

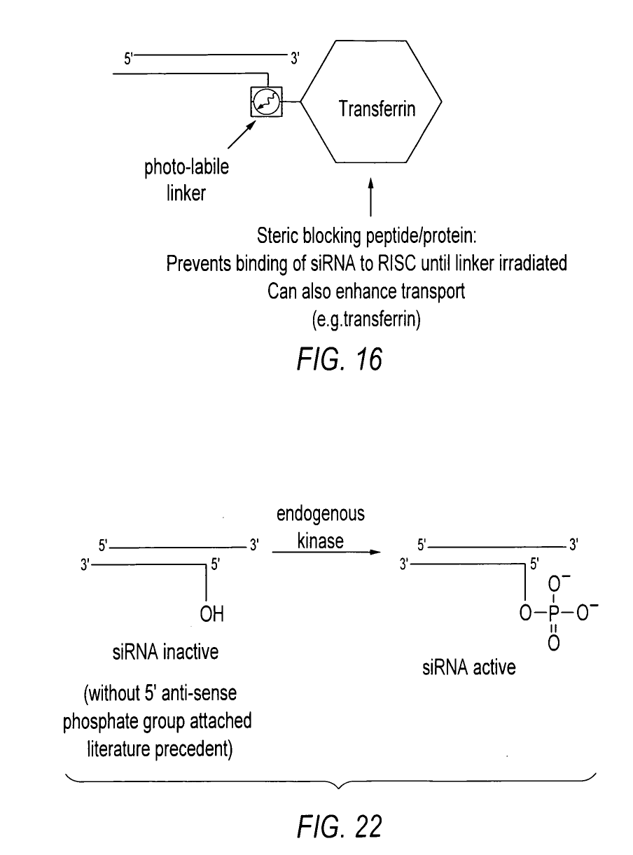

One such method will be herein described for exemplary purposes. In this method (the “Hydralink” method), the ultimate linkage between siRNA and protein/peptide is made via a bis-aromatic hydrazone linkage that forms between two moieties, a benzyaldehyde and a 2-hydrazinopyridyl link. These two groups are attached to the siRNA and protein, and they react spontaneously in water at neutral or slightly acidic pHs to form a stable hydrazone linkage. The benzyaldehyde moiety can be introduced into the cleaved, deprotected and annealed siRNA containing a single amino group at the 5′ terminus by simple acylation using the NHS ester of the benzaldehyde (FIG. 16). The incorporation of the acetone protected hydrazinopyridyl moiety into the protein/peptide can be approached in two different ways depending on the nature of the reactant.

If the reactant is a peptide (or a series of peptides of increasing bulk), the hydrazino group can be incorporated during solid phase peptide synthesis at the N terminus of the peptide, using the available fmoc protected amino-acid. Thus only a single peptide will ultimately be coupled to a single siRNA helix. This is not an option if large proteins are to be examined as they are not easily accessible synthetically. In this case, the hydrazino group will be incorporated through reaction of the native protein. Clearly, the stoichiometry of such a reaction is much more difficult to control, and it is likely that multiple adducts with available protein amines will form. After conjugation, the final conjugate may have several siRNAs attached to a single protein.

Again, the aim of these modifications is to introduce increasing bulk to the specific location of the 5′ phosphate of the siRNA duplex, in an attempt to disrupt the interaction of siRNA with the RISC. It may be that a very small adduct to this terminal amine will completely abrogate RNA interference, so we have described the inclusion of proteins as a limiting case of steric bulk. A significant concern is that the addition of a large protein to the terminus of an siRNA may cause a decrease in transfection efficiency. While this may be the case, there are some compelling proteins/peptides that may function to both sterically block siRNA action (until cleavage of the photo-labile linker) and also facilitate the transport of the siRNA into target cells.

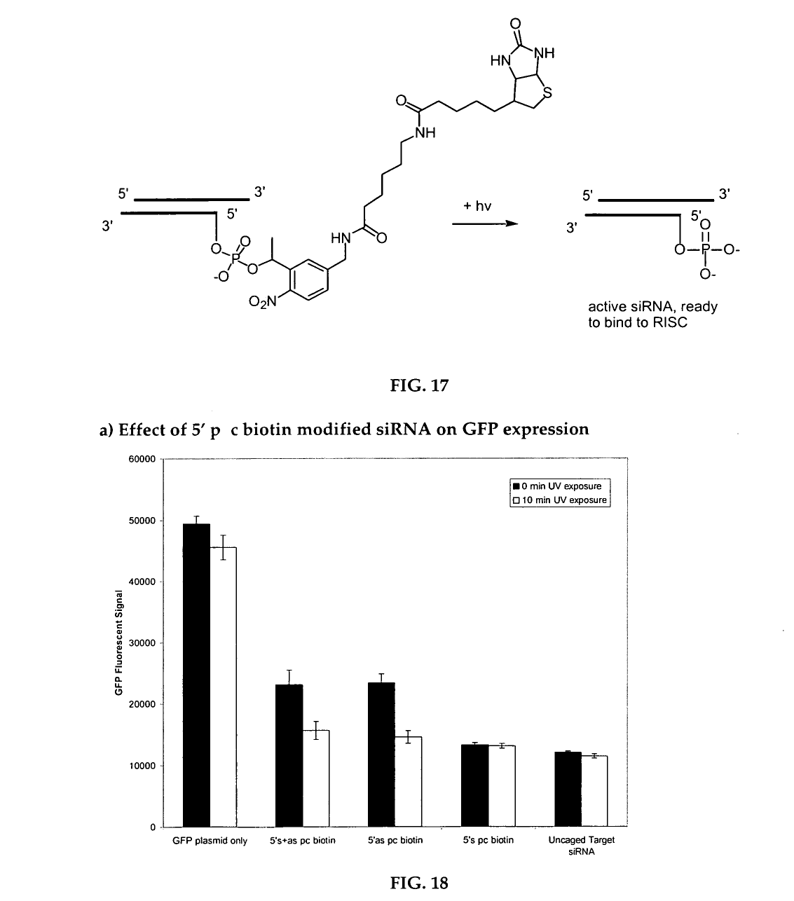

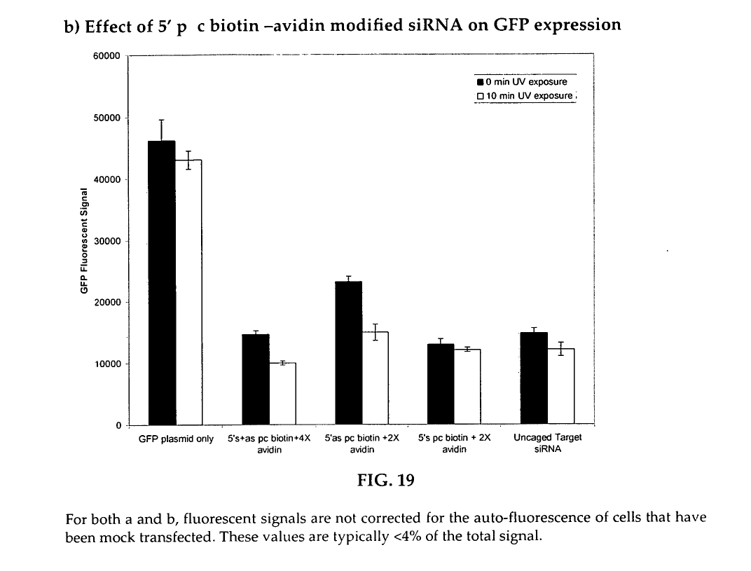

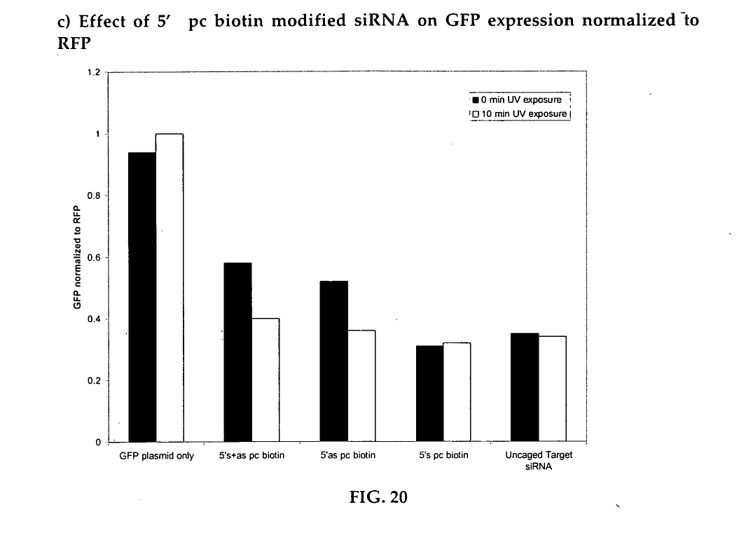

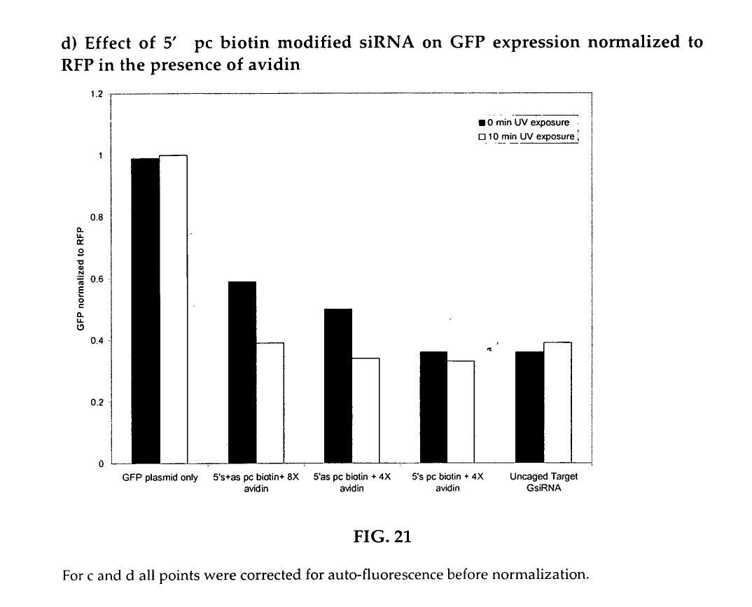

Turning to FIGS. 17-21, data is provided for the embodiment wherein the 5′ phosphate on the antisense strand of siRNA is modified to add bulk. From the results with the photocleavable amine group linked to the 5′ phosphate of the antisense strand of the siRNA, it was found that modifying the 5′ phosphate of the antisense strand was not enough to completely abolish the activity of siRNA in the absence of light. While this was inconsistent with some previous studies, the requirement of a free 5′ phosphate on the antisense strand of the siRNA as being critical for the activity of siRNA was still ambiguous as some more recent studies pointed out that some siRNA sequences retained their gene silencing activity even if the siRNAs contained 5′ modifications on the antisense strand.

It was hypothesized that it was possible that different modifications may have varying abilities to block the activity of siRNA. The ability of 5′ photocleavable biotin modification on the antisense strand of the siRNA to annihilate the activity of siRNA in the absence of light was tested. Moreover, although some research pointed out that modifications on the 5′ end of the sense strand of the siRNA did not have any effect on its activity, given the ambiguity in the field it was decided to test 5′ photocleavable modifications on the sense strand of the siRNA and modifications on both the 5′ sense and 5′ antisense ends of the siRNA.

Finally, it was also reasoned that having a large moiety such as avidin conjugated to the 5′ ends of the siRNA strands may make the siRNA strand inconspicuous to RISC thereby completely blocking its activity in the absence of light irrespective of the necessity of having a free 5′ phosphate on the antisense strand of siRNA for its activity. To test this hypothesis the cells with both siRNA having 5′ pc biotin on one or both strands and avidin were incubated. The hypothesis was that in the absence of light the large avidin group would make the siRNA inaccessible to RISC thereby rendering it inactive and irradiation would release the free siRNA ready to interact with RISC and silence the target gene. It was anticipated that it might be difficult to transfect siRNAs conjugated to avidin because of the bulk imparted to the siRNA by the large avidin groups attached to them. To test the activity of 5′ end modified siRNAs containing either photocleavable biotin modifications or both photocleavable biotin conjugated to avidin, the siRNA sequence targeting GFP was used and both GFP expression levels alone and GFP expression normalized to RFP were observed.

Still another embodiment contemplates steric blocking of 5′ phosphate with transport enhancing proteins. Previous embodiments introduce bulky substituents onto photo-labile linkers that are attached to the 5′ phosphate group of siRNA and in so doing, effectively block the ability of the siRNA to interact with the RISC. This in turn will completely block RNA interference until irradiation has released the active phosphate and removed the blocking group. The instant embodiment includes expanding the role of this bulky peptide/protein moiety from simply blocking RNA interference, to enhancing the tranfection of siRNA.

One of the key limitations of chemically generated siRNA is the need to use transfection agents to effectively introduce the nucleic acid into cells. These are typically cationic lipids such as lipofectamine, which are able to bind to nucleic acids, quench their negative charge and provide enough lipophilicity to allow transport across the lipid bilayer. They are effective in in vitro settings but have limiting toxicities that make them challenging to use for in vivo use. In vivo use is of course one of the potential applications of siRNA, beyond its great power as a tool for biochemical analysis: as a therapeutic to control errant gene expression. One such protein that may be suitable in the dual role of steric blocker and transport enhancer is transferrin. Another class of suitable dual role proteins are the membrane permeabilizing peptides.

Transferrin is an iron transport protein that has been demonstrated to be an effective tool for transfection of molecules including oligonucleotides into cells. This process has been dubbed “transferrin-fection.” It is driven by the recognition and uptake of transferrin by the transferrin receptor. Oligonucleotides that are conjugated to transferrin are also transported and have been observed to exert biochemical effects, such as expression of genes on transfected plasmids. Transferrin therefore is an ideal candidate to be used to block the 5′ phosphate of an siRNA. At 80 kD for the apo enzyme, it has a significant chance of sterically blocking the siRNA/RISC interaction. In addition, it can enhance the uptake of the siRNA through the already characterized “transferrin-fection” path (FIG. 22).

Finally, the attachment of a transport protein like transferrin can help alleviate a specific problem: In siRNAs that have a single photo-labile modification, inadvertent stray light can release active siRNA in advance of experimental irradiation. If these siRNAs are transfected by normal transfection agents, there will be a significant zero time RNA interference effect. However, if the method of transfection is through a protein linked via the photo-labile group, then any siRNAs that have be inadvertently irradiated will not be transported, because the protein will no longer be connected. This will potentially limit this base level of RNA interference caused by early inadvertent photo-cleavage.

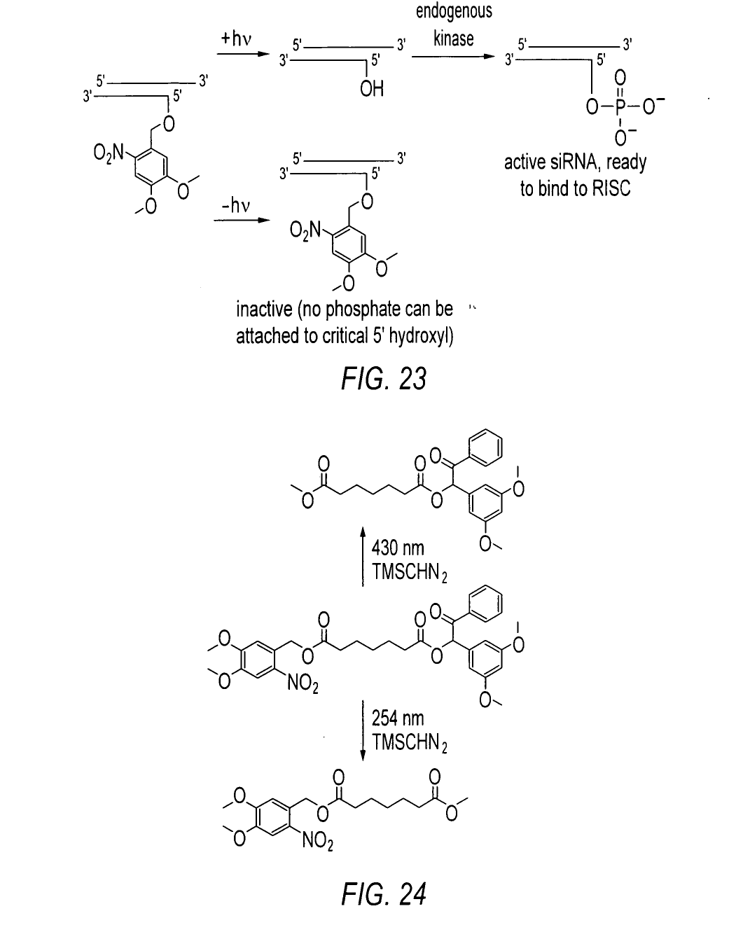

Yet another embodiment contemplates specific blocking of the 5′ hydroxyl group. Previously discussed siRNA modifications will provide insights into the nature of the structural requirements for RNA interference. However, it is also possible to take advantage of requirements already determined from biochemical analysis of RNA interference by siRNA. When dsRNA is processed by Dicer into siRNA duplexes, the 5′ termini are left with phosphate groups attached. Without these phosphate groups, the siRNA can not be incorporated into the RISC, and RNA interference can not take place. Exogenously added siRNAs typically are synthesized without 5′ phosphates. However, an endogenous kinase is able to add phosphates to them, which then allows them to enter into the RISC (FIG. 23).

The 5′ OH then appears to be a useful point of modification to confer photo-control to RNA interference. Blocking this hydroxyl with a photo-labile group should completely prevent endogenous kinase action, and therefore prevent incorporation of the siRNA into the RISC. Upon irradiation, the hydroxyl should be released, modified by the kinase, and the now-activated siRNA incorporated into the RISC, followed by RNA interference (FIG. 24).

The simplest iteration of this is to use the DMNPE group previously described and used for the random blocking of the siRNA backbone. RNA will be synthesized either commercially or on a shared resource ABI 394 using standard phosphoramidites protected with 2′O t-butyl dimethyl silyl (TBDMS) protecting groups. After the terminal monomer has been added, the dimethoxy trityl group protecting the 5′OH will be removed. To the resin bound RNA will be added the diazo form of DMNPE (generated as before by action of MnO2 on the precursor hydrazone), which will react with the 5′OH to form the photo-protected form. At this point in the synthesis, all other potential nucleophiles in the oligonucleotide will be protected, so only the 5′OH should be modified.

The resultant caged RNA will be cleaved from the resin using standard ammonium hydroxide cleavage conditions. The 2′OH protecting groups will then be removed using TBAF. The oligonucleotide will be analyzed using HPLC and/or PAGE to assess purity and, if necessary, purification will be performed. Because of the UV lability of the 5′ OH protecting group, care will need to be taken during purification. For example, the technique of UV-shadowing may cause extensive cleavage of the photo-labile group during PAGE purification of the oligonucleotide. One way around this difficulty is to mask portions of the gel during UV shadowing to preserve the final oligonucleotides and prevent UV exposure. Adjacent, unmasked regions of the gel can then be shadowed and used as guides to cut the appropriate band from the gel in the masked regions. In the inventors' experience, siRNA oligonucleotides made using the 2′ACE protecting chemistry commercialized by Dharmacon are quite pure as synthesized. This may obviate the need for extensive purification. There is precedence from the literature for the stability of the ortho-nitro benzylic group bound to an RNA hydroxyl during the cleavage and deprotection steps required of 2′ TBDMS protected RNA synthesis. It has been shown that a chemically synthesized RNA, modified on a 2′OH with an ortho-nitro benzylic group could be successfully cleaved from the resin using standard conditions to yield the target 2′ blocked oligonucleotide. This suggests that the 5′ OH protected oligonucleotides will be similarly stable.

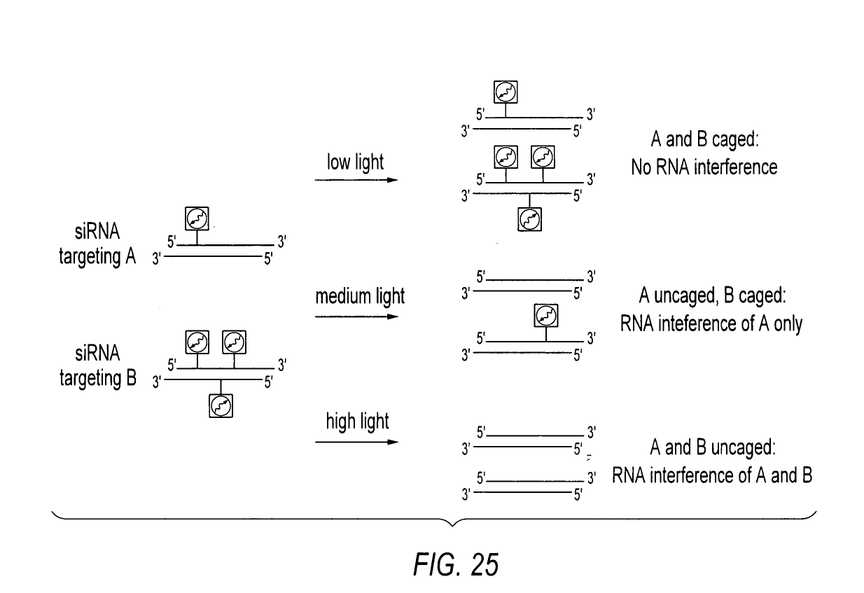

Another embodiment contemplates differential caging to allow differential control of two genes. For any applications of LARI, the ability to control the expression of two or more genes would provide greater power and flexibility. Conceptually, the most direct solution to this problem would be the use of completely orthogonal photo-labile groups, allowing for a different specific wavelength range to deprotect each different siRNA. Although this is an active area of research, only one such pair of photo-labile groups is known to the inventors, as illustrated in FIG. 25. Although these are compelling, they are not completely orthogonal and one requires short UV to deprotect, thereby introducing likely toxicity. As new pairs of photo-labile groups are developed with better compatibility with biological systems, their application may be useful to allow differential control of two genes. Despite the current lack of such groups, there are other approaches that potentially can allow differential control.

One potential approach to the differential control of two different genes relies on differential modification of siRNAs with photo-labile groups using previously described approaches for caging siRNA. As we gain an understanding of the minimum number of caging groups required to block RNA interference and, in addition, understand the dependence on the size of these groups, it should be possible to allow differential control of two different genes.

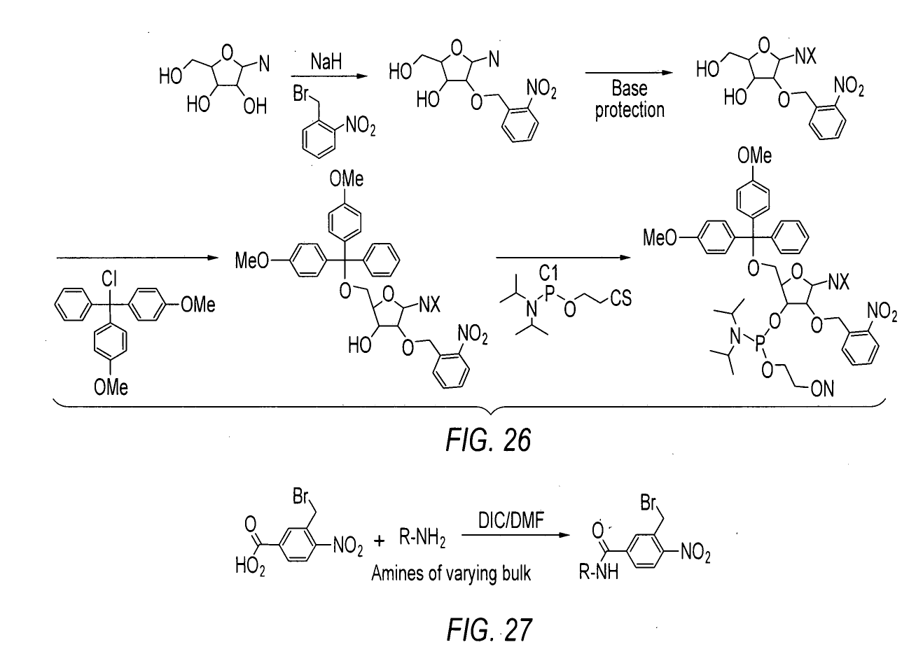

The rationale behind this approach is illustrated in FIG. 26. Given two different genes A and B that are to be differentially down-regulated via RNA interference, two siRNAs that target them are prepared. siRNA A is modified with the minimum number of groups that will completely block RNA interference. siRNA B is modified with a larger number of groups. When co-transfected into cells, these two will allow the two genes to be down-regulated separately. In a “no-light” situation, both gene A and B will be expressed normally. In a medium light exposure situation, siRNA A will be completely deprotected and will down-regulate gene A. However, siRNA B, having a larger number of caging groups will not be completely uncaged, and will therefore be unable to affect expression of gene B. In a high light exposure situation, both siRNA A and siRNA B will be completely uncaged, and both will be able to inhibit the expression of their target genes.

Note, as described, this will only allow the expression of gene A to be reduced independent of gene B, not vice versa. If there is a need to be able to reduce expression of gene B independent of gene A expression, it will be necessary to use a second set of siRNAs with the opposite differential modification levels in a separate experiment. The two genes we will use for testing this approach will be GFP and RFP, both of which will be introduced by transfection of the appropriate plasmids. The use of two fluorescent proteins will allow us to utilize the micro-plate scanning method we have already established in our laboratory to rapidly assess level of both genes.

The invention also contemplates that photo-control of RNA interference by modified siRNA will be sensitive to the sequence position of photo-labile group modification.

One embodiment includes specific blocking of a 2′ OH group and examining dependence of sequence position. Two broad classes of approaches for caging siRNA have been described: random caging of the backbone, and specific caging of key individual termini (5′ OH and phosphates). In addition to these specific locations, the inventors believe that there will be key positions along the length of the siRNA that are needed for siRNA recognition by the RISC. Ideally, the smallest photo-labile addition is sought that will have the largest impact on siRNA/RISC interaction, and therefore RNA interference. Identifying this “ideal” site may allow for the complete caging of an siRNA with a single group. This will then permit the absolute minimum level of irradiation to completely deprotect the siRNA. To this end, it is contemplated that photo-labile groups may be used to block the 2′OH of nucleotides in an siRNA duplex. These will be incorporated at specific sequence positions by using a phosphoramidite with 2′ OH ortho-nitro benzylic protection.

The adenine phosphoramidite has already been synthesized and characterized. It is synthesized in a straightforward fashion from adenine (FIG. 27). Adenine is first modified on the 2′ OH position using ortho nitro benzyl bromide. This is the photo-labile group. Subsequently, after benzoyl protection of the exocyclic nitrogen of the base, the 5′ OH is modified with a DMT group, and finally the 3′ OH converted to the phosphoramidite using 2-cyanoethyl-N,N-di-isopropyl amino chlorophosphite. This phosphoramidite can be incorporated into RNA oligomers during standard solid phase synthesis (2′ TBDMS protection was used for the non-caged RNA positions). Characterization confirmed that the modified nucleotide was stably incorporated into the oligonucleotide chain.

The GFP targeting siRNA has five adenines in the sense strand and four in the anti-sense strand. Initially four oligonucleotides that incorporate the photo-labile group in each of the four adenine positions in the anti-sense strand of the target siRNA are obtained. These will be incorporated into siRNAs and tested using the GFP expression system described herein. The invention contemplates that there will be a positional dependency on the effectiveness at blocking RNA interference. Focus is on the anti-sense strand in the first iteration of this approach because there is significant precedence that the RNA interference is more sensitive to modifications in the anti-sense strand.

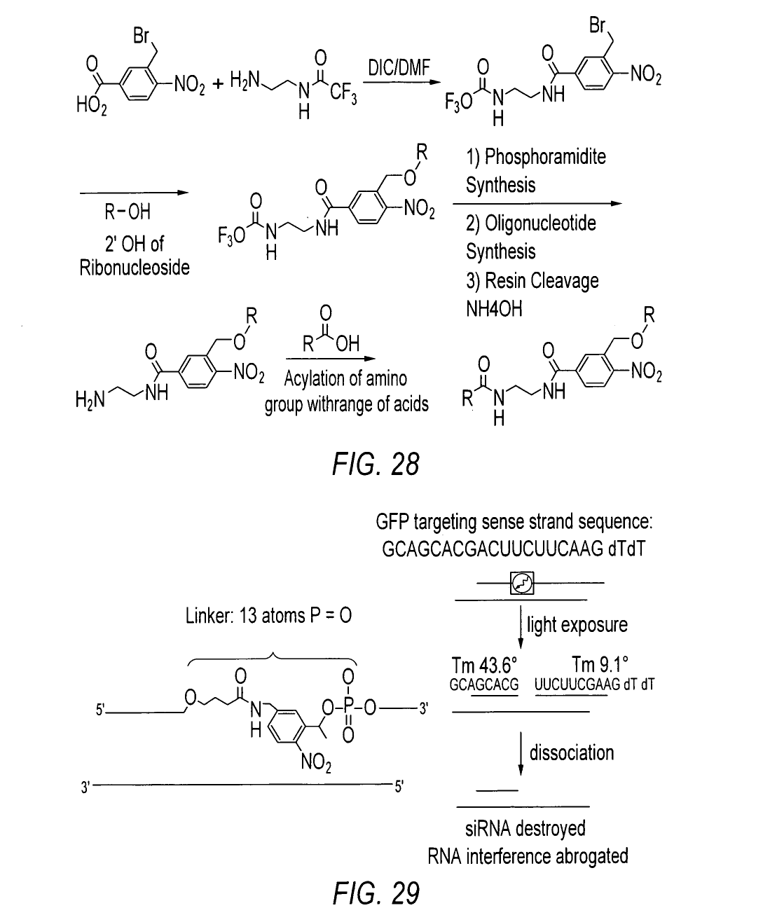

As in previously described iterations in this application, the importance of steric bulk at the 2′ OH position may also be contemplated by modifying the photo-labile group. While sequence position is of likely importance, it is anticipated that the size of the group may be important. To explore this possibility, a set of varying sized ortho-nitro benzylic protecting groups may be devised, using the commercially available benzyl-bromo starting material shown in FIG. 28. This material can be activated with DIC and reacted with a range of commercially available amines to make a series of caging agents of increasing bulk. This compound, when activated by DIC, will selectively and efficiently react with an amine nucleophile at the carboxylic acid position, and not the benzylic position, to give 90% conversion to the amide. These modified benzyl-bromide compounds can then be incorporated into the phosphoramidite synthesis in the analogous position as the unmodified ortho-nitro benzyl bromide.

A further and more flexible iteration of this approach is to react the carboxylic acid with the hemi-trifluoro acetamide protected diamine in FIG. 29. This will provide a protected amine that is stable to the conditions of solid phase oligonucleotide synthesis but will be cleaved by the ammonium hydroxide used for the final cleavage from the resin. Trifluoro-acetamide protection is a typical group used to protect primary amines in linkers used in oligonucleotide synthesis. After synthesis and cleavage from the resin, this amino group can be further modified through acylation with any of the approaches described previously including whole proteins. This will permit even greater flexibility for identifying adducts that will completely block RNA interference.

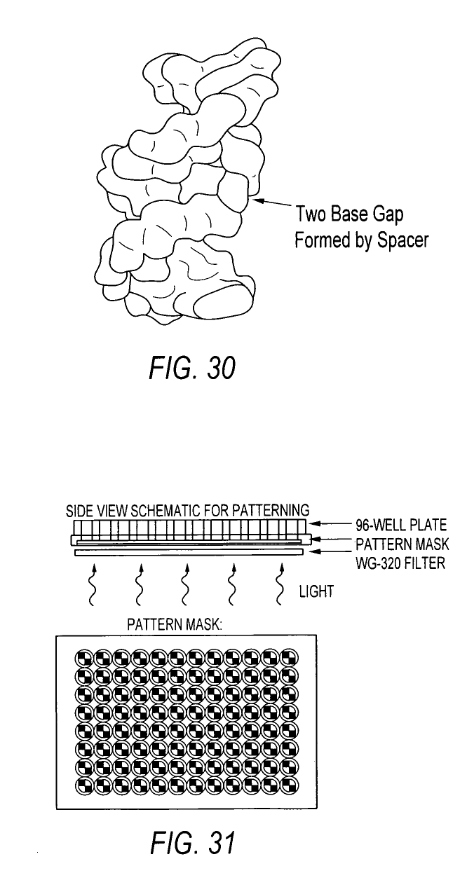

Another embodiment contemplates a sense strand photo-labile linker approach. As opposed to the methods described previously, which rely on the blocking of the siRNA with photo-labile groups, the sequence effects of a photo cleavable linker placed in the middle of the sense strand is also contemplated by the invention. It has been observed that the sense strand of siRNA is much more tolerant towards modification than is the anti-sense strand, and can actually tolerate bulges. This is to be expected, as the anti-sense strand is the one that ultimately directs the binding of the target mRNA in the RISC. It is anticipated that an appropriate linker will be tolerated in the sense strand of the siRNA and allow RNA interference to take place. Upon irradiation, the sense strand will be split into two smaller strands, at least one of which will have a Tm below room temperature and will thus dissociate, destroying the siRNA (FIG. 30). Therefore, light exposure will eliminate RNA interference, as opposed to induce it. In theory then one could control two different genes with light. In cells that were transfected with a blocked siRNA that targeted gene A and with a sense-strand photo-labile linker siRNA that targeted gene B, un-irradiated cells would have full expression of gene A and reduced expression of gene B. In irradiated cells, gene A would be reduced in expression and gene B would have full expression. This approach would cover only a very small subset of the possible experiments that could be performed by a truly orthogonal photo-labile protecting group system. Despite that, it could have additional advantages over those blocking methods described previously. Specifically, it could be a solution to the “incomplete caging” phenomenon observed with random backbone caging.

Summarizing those results, random caging of the siRNA backbone two non-ideal situations can occur: In samples that have not been highly substituted with photo-labile groups, RNA interference is not fully blocked. In samples that are highly substituted, RNA interference is fully blocked, but cannot be fully released upon irradiation. The sense strand photo-labile linker approach could remedy this, because it would be expected that a single photo-cleavage event per siRNA duplex will completely destroy its ability to cause RNA interference. Of course, its ability to cause RNA interference before exposure will hinge critically on its ability to mimic a native siRNA duplex.

Still other embodiments contemplate an approach that includes specific designs for a linker as the photo-labile linker. There are two issues that are key for the design of the photo-labile linker: the ability of the linker to allow active siRNA duplex formation, and ability of the linker to cause the collapse of the duplex after photo-cleavage. Both issues in the selection of the first generation of linkers have been analyzed. While the sense strand of siRNA is very tolerant towards modifications, it will be critical to use a minimally perturbing linker to maximize the chance that the siRNA duplex that incorporates it will still be fully active for RNA interference. FIG. 30 depicts the first selection. It utilizes the same ortho-nitro benzylic motif that forms the basis for the other modifications described in this application. It is depicted in the figure as the commercially available phosphoramidite that gets incorporated into a growing oligonucleotide chain at the 5′ hydroxyl. The terminal dimethoxy trityl group is deprotected and another nucleotide can then be added to the chain. The entire distance between the phosphorus atom of the linker and the phosphorus atom of the next nucleotide is 13 atoms. This compares favorably with the number of atoms found in two base pairs, 12. This suggests that it should be able to bridge the gap that would normally be occupied by two bases. This supposition is supported by molecular modeling. Two middle bases were removed from a crystal structure of duplex oligonucleotides (pdb 1EFS) and the linker built in, using the MMFF force field. Minimization produced a structure with no significant strain or bad torsions in the linker, suggesting, at a minimum, that the linker can be accommodated without distorting the helix (FIG. 30).

The second design issue is the ability of the smaller pieces to dissociate after photolysis of the linker. The exact position of placement of the linker in the oligonucleotide will determine this. The sequence of the sense strand of the GFP-targeting siRNA is shown in FIG. 30. Also are depicted the calculated Tm values for the remaining oligonucleotides after cleavage of a two base spanning photo-labile linker. The 3′ side of the oligonucleotide is AU rich, so it is advantageous to make that portion as long as possible while still having a Tm<room temperature. The first attempt will be the linker placement indicated, as it results in the creation of two oligonucleotides, one with 8 bases and a 43.6° C. Tm, and one with 11 bases (including a 2 base overhang) and a 9.1° C. Tm. This is an appealing choice of position, as one of the resultant oligonucleotides is both substantial (˜half of the duplex) and likely to dissociate (Tm<<rt).

The linker described above is a single example, and lends itself to multiple iterations. For example, the sequence position placement of the linker in the strand can be varied, as there may be differential effects on ability to function as an siRNA and on the ability to abrogate this function upon irradiation. In addition, it may be necessary to further tune the linker, and adjust the number of methylenes between the amide nitrogen and the DMT protected oxygen. If necessary, this should be fairly straightforward to do, as the syntheses of related monomers have been described in the literature and numerous carboxy alcohols are commercially available.

The instant invention contemplates the use of light activated RNA interference (LARI) to analyze biological systems. The invention contemplates a photo-labile system that will completely prevent RNA interference by siRNA until irradiation takes place, at which point the siRNA will be completely active. While the applications are limitless, three areas are especially fertile. The applications are in three areas: 1) Patterning of gene expression 2) Examining the fundamental kinetics of the RNA interference phenomenon and 3) Collaborative studies in developmental biology.

A first application is the patterning of gene expression. The invention contemplates making defined patterns of gene expression in cultures of live cells. The potential applications of such methods range from nano-technology (informational storage, neural circuits etc.) to tissue engineering. Selectively controlling specific gene expression in specific spatial regions may allow the differential control of cell development, leading to defined arrays of different cell types, thus permitting the engineering of tissues.

The key pieces of such a system are a method for masking regions of cells as well as a method for complete toggling of RNA interference. Initial attempts at pattern masking have been made, but the “contrast” in differential RNA interference to date is not sufficient to generate a strongly visible pattern. One mask used is the pattern shown in FIG. 31 that is laser printed onto a thin film and then adhered to the outside of the bottom of a 96-well plate well. The plate is then exposed from below using the lamp previously described to effect uncaging of siRNA in regions of light, and to maintain caging in the shadowed regions of darkness. The design of the mask is meant to allow the same pattern to be observed on all scales (i.e. four quadrants of alternating intensity) to allow an assessment of the resolution of the ultimate cellular pattern. When examined using a microscope, these laser printed patterns are fairly rough at the 50 μm scale, making them sufficient for rough patterning, but not for cell resolution. It was found that film patterned with a slide printer can have a resolution of 5 μm which is approaching cellular resolution. The invention contemplates that standard methods of masking used in semi-conductor synethesis may prove particularly valuable in this endeavor. When a method to completely toggle siRNA from 0 to 100% RNA interference is achieved, generation of the indicated pattern in alternating zones of high and low GFP expression is contemplated.

The invention contemplates examination of RNA interference kinetics. Despite the importance of RNA interference, there are significant gaps in the collective understanding of the fundamental biochemistry of its action. Accordingly, the invention contemplates utilizing light activated RNA interference to examine the kinetics of RNA interference. Specifically, the invention contemplates an understanding of how rapidly RNA interference initiates once siRNAs enter the cell. The results of this analysis could have implications for the pharmaco-kinetics of therapeutic applications of siRNA. This kind of question cannot be easily answered at present, as the transfection process itself takes an indeterminate amount of time. However, with caged siRNA, RNA interference can be initiated only after the cell has equilibrated with siRNA and the transfection solution removed. This would be initiated by irradiation of the cells to uncage the target siRNA. The rate of change of GFP signal relative to an untreated cell culture could then be monitored. This would then give the very practical limit of the rate at which RNA interference effects initiate, and ultimately dissipate. It is anticipated that there will be a lag time after photo-release, during which time the siRNA is processed and bound to the RISC, at which point mRNA degradation will begin and an decrease in gene expression is observed. This is a single example of an application of light activated RNA interference to analyze fundamental aspects of RNA interference. It is anticipated that it will be a useful tool as researchers in the field seek a more detailed understanding of RNA interference.

The invention also contemplates use in the study of development. One of the most promising applications of the methods pursued herein is in studies of development. Development examines how the fertilized egg develops into a cluster of undifferentiated cells, each of which eventually differentiates and achieves specific function. One of the key classes of proteins which governs this process are morphogens, signaling proteins that can impose a pattern of development on a whole field of cells. They can do this by forming concentration gradients across the growing organism. This gradient can be created by diffusion of the protein from one end of the organism to the other. In turn, cells are sensitive to the concentration of these morphogens, such that their developmental path will alter as critical concentrations are reached. An important point is that there can be more than just two developmental paths and the “decision” to follow one or the other is based on this gradient.

The invention anticipates that light activated RNA interference will allow the spacing, the timing and, most importantly, the level of expression of putative morphogens to be effectively manipulated. This should allow a very efficient probing of their importance by developmental biologists. Preliminary results indicate that the level of protein expression can be modulated by the duration of deprotection irradiation. It potentially can also be modulated via a gradient filter. Development is rooted in the timing of spatial changes in gene expression levels, and LARI should permit an unprecedented ability to manipulate these levels in an effort to understand development.

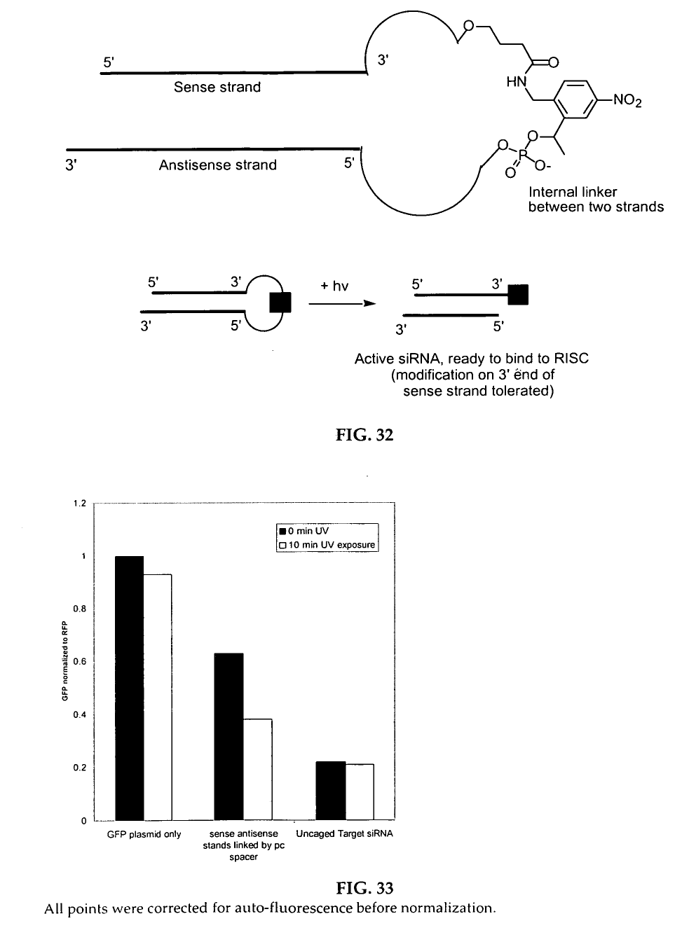

Another embodiment of the invention contemplates designing a hairpin by linking the sense and antisense strands with a photocleavable linker, as illustrated in FIG. 32. This means that the two strands should not be able to unwind completely in the absence of light. Unwinding of the two strands is considered as a critical step in the processing of siRNA by RISC. Secondly, the strain formed in the duplex by an internal linker forming a loop at one end of the siRNA might modify the conformation of siRNA such that it is not recognized by RISC. Irradiation with light should result in severing the link between the two strands allowing them to unwind and be processed by RISC. The siRNA with the internal linker was designed such that after irradiation it would leave a photolabile group on the 3′ phosphate of the sense strand. Almost all modifications on the 3′ sense end of the siRNA seem to be tolerated very well by the RISC and various siRNA sequences with 3′ modifications on the sense strand exhibit gene silencing levels comparable to unmodified siRNAs. FIG. 33 illustrates GFP expression using this strategy.

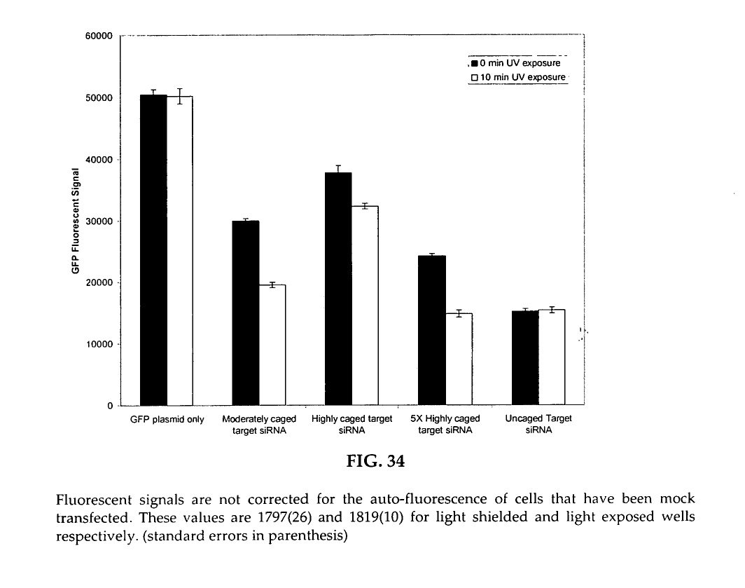

Another embodiment includes delivering a large concentration of the highly caged siRNA. Activity of siRNA can be completely abolished by modifying the siRNA with a large number of caging groups. As illustrated in FIG. 34, increasing the concentration of highly caged siRNA resulted in a commensurate increase of caged/uncaged siRNA that were able to be processed by RISC, thereby resulting in a decrease in the loss of gene silencing activity for even highly caged siRNA. Increasing the concentration of highly caged siRNA simply allowed movement of the window of modulation with light but did not complete control over modulating RNAi with light.

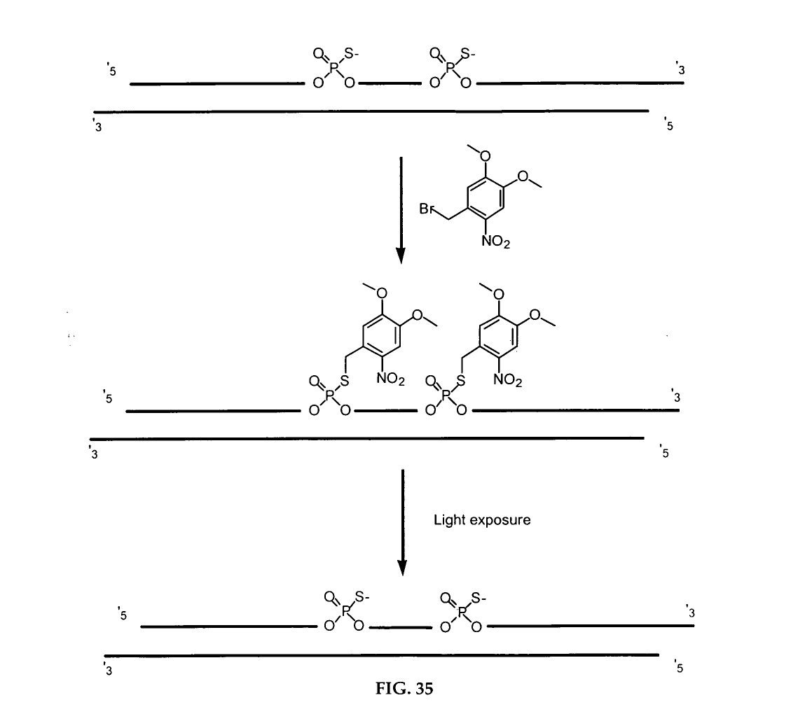

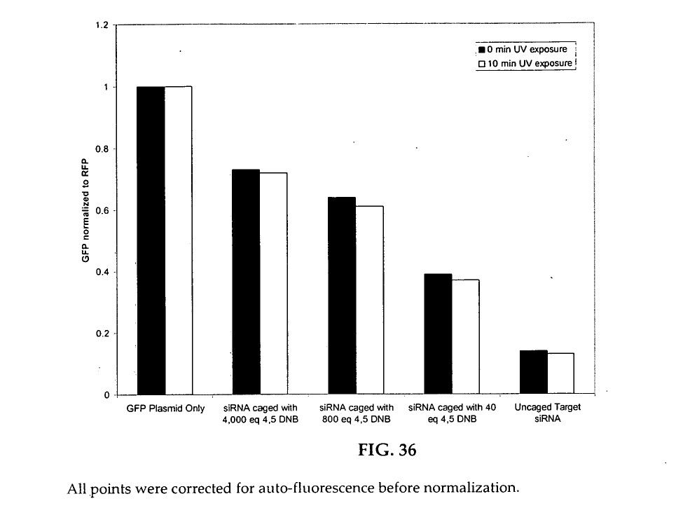

Still another embodiment contemplates targeting key positions on the siRNA using phosphorothioate chemistry. (FIGS. 35 and 36) As illustrated in FIG. 35, an siRNA sequence is included that has phosphorothioate linkages between base nine and ten and base ten and eleven of the antisense strand. To cage the siRNA, 4,5 dimethoxy 2-nitrobenzyl bromide was used. Since only the specific phosphorothioate residues were to be caged, and not other positions on the phosphodiester backbone, the caging reaction was carried out using three different concentrations of 4,5 DNB to determine the exact concentration required to modify the more reactive phosphorothioate residues without modifying any other positions on the siRNA duplex.

Antisense strand containing phosphorothioate linkages was obtained from Dharmacon and reacted with 40,800 and 4000 eqs of 4,5 DNB. Samples were allowed to shake for 30 hours at room temperature and unreacted 4,5 DNB was removed by extraction with chloroform. Extent of caging was determined using the absorbance of the sample at 1=355. Based on this technique, 18%, 22% and 38% of the total phosphates/phosphorothioates on the antisense strand were modified. These modified strands were then annealed with unmodified sense strands and tested using the standard assay.

As illustrated in FIG. 36, increasing the eq. of 4,5 DNB resulted in an increased loss of the ability of the siRNAs to cause RNAi in the absence of light. However similar to the problem faced while caging the 5′ hydroxyl, caging on phosphorothioate residues resulted in an inability to undergo photocleavage upon exposure to the standard amount of light used in previous experiments. It may also be possible that using 4,5 DNB might have resulted in modification at positions other than the phosphodiester backbone (e.g. N on the nucleobases) resulting in the inability to undergo photocleavage with light.

Another embodiment contemplates caging the 5′ OH on antisense strand by using 4,5 dimethoxy-2-nitrobenzyl chloroformate.

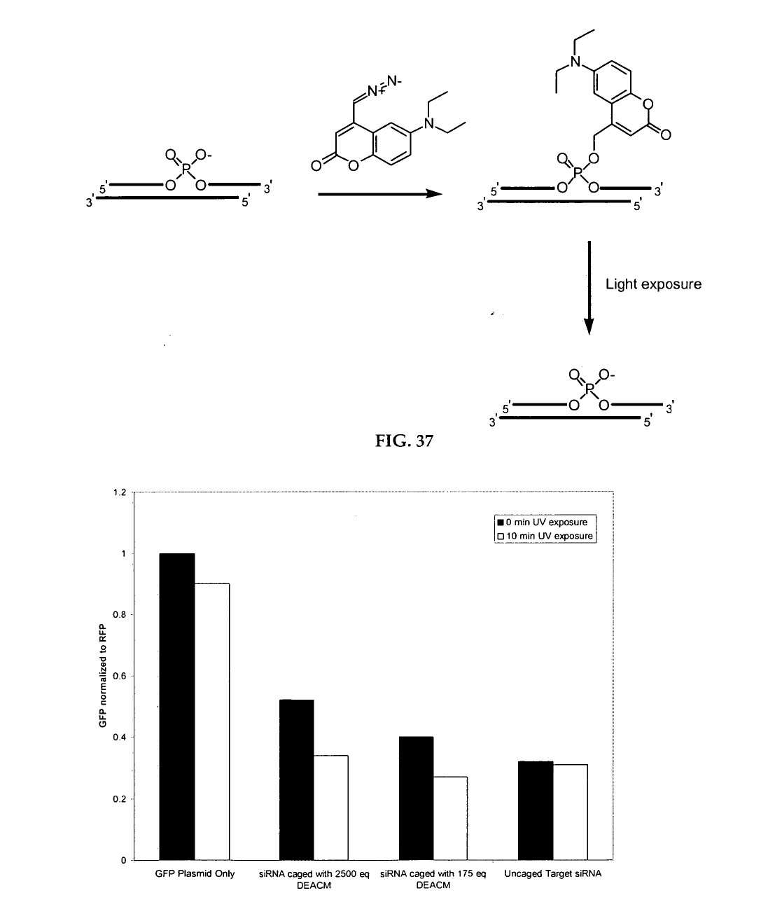

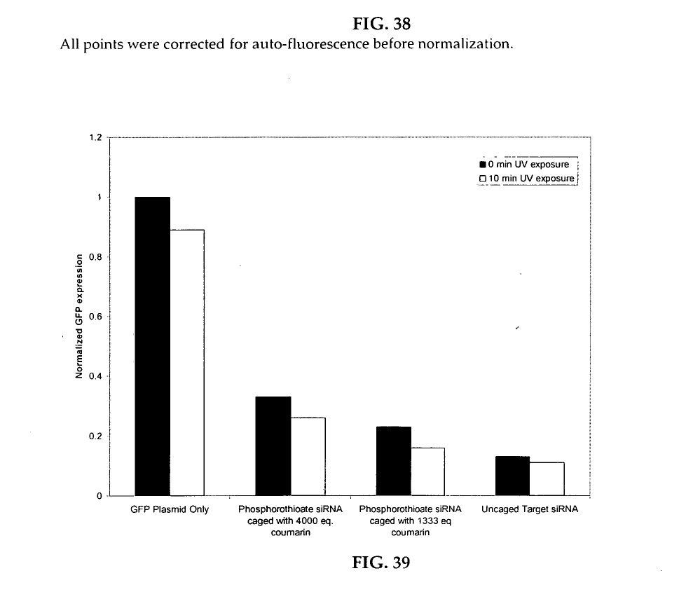

Turning now to FIGS. 37-39, another embodiment includes modifying siRNA with the [7-(diethylamino)coumarin-4yl]methyl (DEACM) caging group. DEACM is a novel caging group that has been used for caging cAMP and 8-bromo-substituted cyclic nucleotides. The long wavelength absorption and high extinction coefficients exhibited by DEACM caged compounds could potentially allow the deprotection of caged substrates inside cells under nondamaging light conditions. 7-diethylamino-substituted 4-(diazomethyl)coumarin was synthesized by using a procedure described by Hagen and coworkers. 7-(diethylamino)-4-formylcoumarin was synthesized by selenium dioxide oxidation of 7-(diethylamino)-4-methylcoumarin and purified by column chromatography. The corresponding 7-(diethylamino)-4-formyl coumarin tosylhydrazone and 4-(diazomethyl)-7-(diethylamino)coumarin were synthesized as previously described in literature. All compounds synthesized were characterized by NMR. 2500 eq. and 75 eq. of this caging compound in DMSO were reacted with siRNA present in water. The excess caging material was removed by chloroform extractions and precipitation with alcohol.

Complete caging was not achieved with the Hagen compound even by using a very high concentration of DEACM. However, caging was effected with some promising efficacy. We assumed that exposure to ambient light might also result in deprotection since these caging compounds showed an absorption maxima around 402 nm. We decided to test the effect of these coumarins on phosphorothioate containing siRNAs since these phosphorothioates may be more reactive than the regular phosphodiester backbone and we could get a more complete caging. We used the same phosphorothioate sequence used previously with 4,5 DNB to target specific positions on the siRNA strand. The phosphorothioate linkage containing siRNA was reacted with 1333 eq. and 4000 eq. of DEACM and excess free DEACM removed by extracting with chloroform. Using UV absorbance and the extinction coefficient of the DEACM caged ATP at 402 nm, we found the caging efficiency to be 16.47% and 3.9% in the 1333 eq. and 4000 eq. conditions respectively. Activity of these samples was tested using the normalized GFP expression assay.

It is possible that a higher caging efficiency than the 16.47% obtained with the conditions above might allow a full toggling of the RNAi effect. Testing different ratios of DMSO:water to carry out the caging reaction, different solvents, temperature and other conditions might allow a complete caging of the siRNA with the DEACM group. As expected, deprotection from the phosphorothioate containing sequence seems to be more challenging than deprotection from the native siRNA. Due to the absorption maxima of around 402 nm, special care might be required while handling this compound.

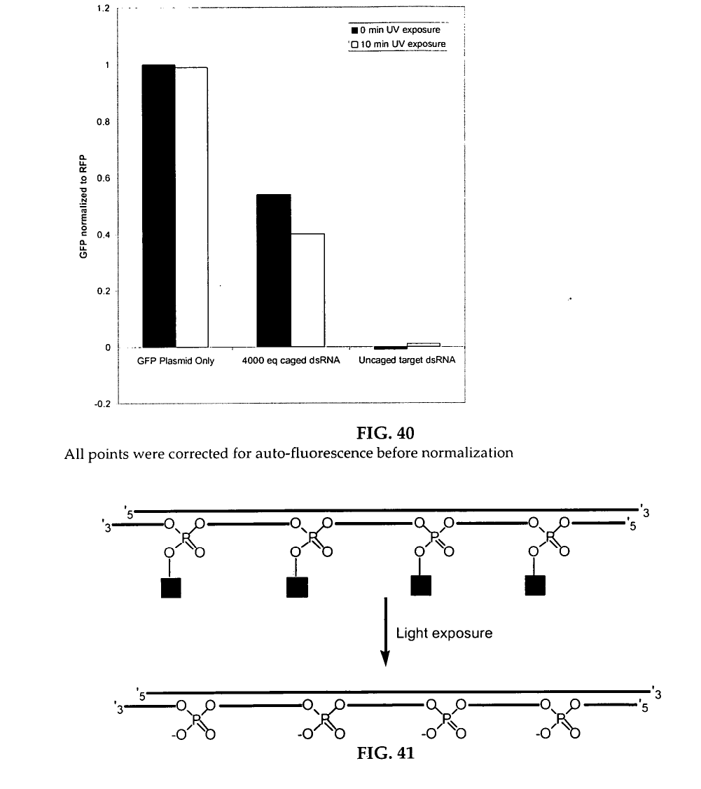

Turning to FIG. 40, another embodiment contemplates caging dsRNA which needs to be processed by both Dicer and RISC before exhibiting RNAi effect. The lag time of around 40 hours displayed by siRNA sequences before exhibiting their full RNAi effect may become a limitation in some studies where faster gene silencing may be required to study the desired effect. A potential solution to this problem is to use synthetic RNA duplexes, 25-30 nucleotides in length, which not only show faster gene silencing as compared to siRNAs but also function at much lower concentrations. (˜100 fold lesser than the corresponding siRNA sequences) Studies of the kinetics of gene silencing using dsRNA targeting GFP revealed an ability to knock off around 90% of the target gene, 15 hours after transfection as opposed to the 42 hour period required to knock off around 70% of the target gene using conventional siRNA sequences. The enhanced potency of the longer duplexes is ascribed to the fact that they are substrates of the Dicer endonuclease, thereby, directly linking the production of siRNAs to incorporation by RISC.

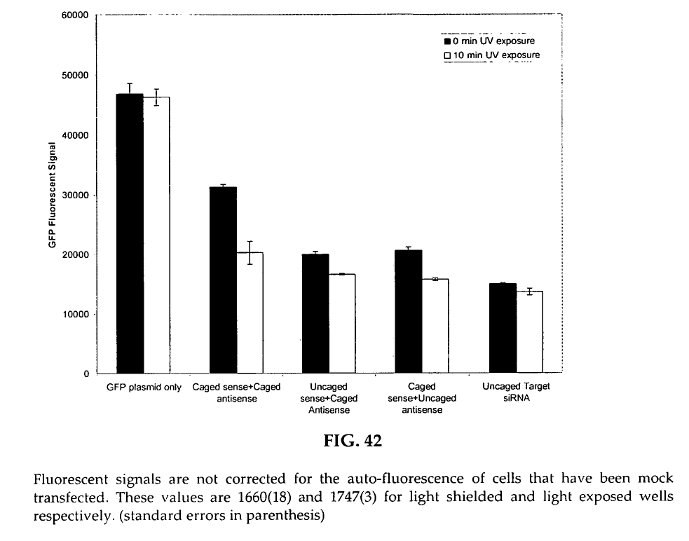

Turning to FIGS. 41 and 42, still another embodiment includes caging either the sense or antisense strand alone, as opposed to caging the entire duplex. Although the precise biochemical mechanism of RNAi is not completely understood, it is believed that after unwinding of the siRNA, the antisense strand of the siRNA is taken up by RISC. The antisense strand of the siRNA then hybridizes with the complementary sequence of the target mRNA through Watson-Crick base pairing and cleaves the target mRNA. We assumed that since the antisense strand of the siRNA is taken up by RISC, the antisense strand of the siRNA may be more sensitive to modifications than the sense strand. The belief was corroborated by studies from some other groups that demonstrated that the sense strand of siRNA was more tolerable to structural and chemical modifications as compared to the antisense strand. It was hypothesized that caging the antisense strand alone might require lesser amount of modification allowing deprotection of the siRNA under non damaging light conditions. To test this hypothesis, sense and antisense strands of the siRNA were caged separately and annealed with their caged or uncaged complementary strands (see below) after completing the caging reaction.

As illustrated in FIGS. 41-42, it seems that caging the sense or antisense strand alone prior to annealing did not result in a species that had reduced silencing abilities as compared to the siRNA annealed prior to caging.

In addition to the use of siRNA, an additional embodiment of the invention contemplates use of double stranded siRNA precursors to effect light activated RNA interference. These RNA duplexes, which are preferably 25-30 nucleotides in length and do not contain a 3′ overhang, exhibit faster gene silencing when compared to siRNAs and also function at much lower concentrations (on the order of 100 fold lesser than the corresponding siRNA sequences). Using these longer precursors also reduces target gene expression by 90% in approximately 15 hours after transfection, as opposed to the 42 hour period associated with the ability of conventional siRNA sequences to reduce target gene expression by 70%. The enhanced potency of the longer duplexes is ascribed to the fact that they are substrates of the Dicer endonuclease, thereby directly linking the production of siRNAs to incorporation by RISC. All of the strategies described herein for modifying siRNA with photo-labile groups can be applied to these double stranded siRNA precursors as well. Besides the advantages of faster silencing and higher potency offered by these long duplexes, they may be much more sensitive to photo-labile modifications as compared to native siRNA duplexes, because the precursors have to be processed by at least two enzymes, Dicer and the RISC.

While particular embodiments of the invention have been described herein, it will be appreciated by those skilled in the art that changes and modifications may be made thereto without departing from the invention in its broader aspects and as set forth in the following claims.

What is claimed is:

1. A method for controlling the spacing, timing and degree of gene expression comprising:
selecting a target mRNA;
obtaining siRNA corresponding to the target mRNA;
modifying the siRNA with at least one photo-labile group to inhibit RNA interference;
introducing the modified siRNA into a cell; and
selectively irradiating the cell with light having a predetermined wavelength.

2. The method of claim 1 further comprising modifying a phosphate backbone of the siRNA with the photo-labile group.

3. The method of claim 1 further comprising modifying one or both of a 3′ hydroxyl group and a 5′ hydroxyl group of the siRNA with the photo-labile group.

4. The method of claim 1 further comprising modifying one or both of a 3′ phosphate group and a 5′ phosphate group with the photo-labile group.

5. The method of claim 1 further comprising modifying the 2′ OH groups of the siRNA with the photo-labile group.

6. The method of claim 1 further comprising replacing nucleotides in one or both strands of siRNA with a photo-cleavable linker.

7. The method of claim 2 wherein the photo-labile group is further conjugated to other molecules to confer one or both of steric bulk and membrane transport ability.

8. The method of claim 3 wherein the photo-labile group is further conjugated to other molecules to confer one or both of steric bulk and membrane transport ability.

9. The method of claim 4 wherein the photo-labile group is further conjugated to other molecules to confer one or both of steric bulk and membrane transport ability.

10. The method of claim 2 wherein the photo-labile group is further conjugated to other molecules to confer one or both of steric bulk and membrane transport ability.

11. The method of claim 1 further comprising linking a sense and an antisense strand of the siRNA to create a hairpin

12. The method of claim 1 further comprising delivering a large concentration of highly caged siRNA to the cell.

13. The method of claim 1 further comprising modifying predetermined positions on the siRNA with phophorothioate chemistry.

14. The method of claim 1 further comprising modifying a 5′ OH on an antisense strand with 4,5 dimethoxy-2-nitrobenzyl chloroformate.

15. The method of claim 1 wherein the photo-labile group is selected from the group consisting of DMNPE, DEACM, Bhc-diazo, 2-nitrobenzyl, 4,5 dimethoxy 2-nitrobenzyl, Alpha-carboxy-2-nitrobenzyl, 1-(2-nitrophenyl)ethyl nitroindoline, 4-methoxy 7-nitroindoline, 1-acyl 7-nitroindolines, 1-(2-nitrophenyl)ethyl ethers of 7-hydroxycoumarins, 7-(alkoxy coumarin-4yl)methyl esters, 6,7-(dialkoxy coumarin-4yl)methyl esters, 6-bromo-7-(alkoxy coumarin-4yl)methyl esters, 7-dialkylamino (coumarin-4yl)methyl esters, p-hydroxyphenacyl, and 6-bromo-7-hydroxycoumarin-4-ylmethyl.

16. The method of claim 1 further comprising selectively irradiating the cell with ultraviolet light.

17. The method of claim 1 wherein the cell is irradiated with ultraviolet light having a wavelength greater than 320 nm.

18. A modified siRNA capable modulating gene expression comprising:
an siRNA that targets a predetermined complementary mRNA; and
at least one photo-labile group attached to said siRNA.

19. The modified siRNA of claim 1 wherein said photo-labile group is selected from the group consisting of DMNPE, DEACM and Bhc-diazo.

20. A modified double stranded siRNA precursor capable of modulating gene expression comprising:
at least 25 nucleotides; and
at least one photo-labile group attached to said siRNA precursor.
